(19) United States
(12) Patent Application Publication
(10) Pub. No.: US 2005/0032784 A1
(43) Pub. Date: Feb. 10, 2005

(54) Combination drug therapy for treating hypertension

(75) Inventors: Carl J. Pepine, Gainesville, FL (US); Eileen M. Handberg, Gainesville, FL (US); Rhonda M. Cooper-DeHoff, Gainesville, FL (US); Peter S. Zilles, Lampertheim (DE); Udo F. Legler, Landau/Pfalz (DE); Robert Altman, Wilmette, IL (US); Jeff Drajesk, Burlington, WI (US); Azmi Nabulsi, Barrington, IL (US); Laura Williams, Gurnee, IL (US)

Correspondence Address: FOLEY AND LARDNER;SUITE 500, 3000 K STREET NW, WASHINGTON, DC, 20007, US

(73) Assignees: University of Florida Research Foundation, Inc.; Abbott Laboratories

(21) Appl. No.: 10/816,461

(22) Filed: Apr. 1, 2004

Related U.S. Application Data
(60) Provisional application No. 60/459,563, filed on Apr. 1, 2003.

(51) Int. Cl. A61K 31/55; A61K 31/549; A61K 31/277

(52) U.S. Cl. 514/223.5; 514/423; 514/521

(57) ABSTRACT

Disclosed are compositions and methods for treating, preventing, and ameliorating hypertension, especially in patients with coronary artery disease, patients at risk for strokes, patients with renal disease, and patients with diabetes or at risk for diabetes. The compositions and methods relate to a combination therapy using three classes of anti-hypertension drugs, including calcium antagonists, angiotensin-converting-enzyme inhibitors, and diuretics. The preferred drugs in the compositions and methods are verapamil, trandolapril, and hydrochlorothiazide.

### CROSS-REFERENCE TO RELATED APPLICATIONS

This application claims the benefit of U.S. provisional application Ser. No. 60/459,563, filed Apr. 1, 2003, which is incorporated herein by reference in its entirety.

### STATEMENT REGARDING FEDERALLY SPONSORED RESEARCH OR DEVELOPMENT

This invention was made with U.S. government support under the National Heart, Lung, and Blood Institute Grant Nos. U01HL64924-02 and N01-HV-68163. The U.S. government has certain rights in this invention.

### BACKGROUND

Cardiovascular disease has become a global burden, manifesting itself in ever-increasing rates of coronary heart disease (CHD), hypertension (HTN), and stroke. In the United States alone, cost estimates for 2003 cardiovascular disease-related health-care expenditures exceed $351.8 billion. The world's population is aging, and although deaths due to acute myocardial infarction (MI) have decreased, the prevalence of obesity, inactivity, and other risk factors is increasing, leading to an epidemic of diabetes and other comorbid conditions.

Hypertension management in patients has become a complex clinical challenge, especially in patients with coronary artery disease (CAD) in which HTN management requires not only lowering blood pressure (BP), but also comprehensive therapy focused on the entire vascular system, with the goal of preserving organ function. Although anti-hypertensive drug therapy may reduce morbidity and mortality, the optimal choice for initial therapy of HTN is uncertain, especially in CAD patients who often are excluded from HTN treatment trials.

Previous trials documented the benefit of lowering BP primarily by using thiazide diuretics, (e.g., hydrochlorothiazide), and beta-adrenergic blockers, but these trials were predominantly done in elderly patients who had isolated systolic HTN and in an era prior to coronary revascularization, organ protection, and other contemporary treatments. When newer anti-hypertensive agents (e.g., dihydropyridine calcium antagonists (DHP CAs), non-dihydropyridine calcium antagonists (non-DHP CAs), and angiotensin-converting-enzyme inhibitors (ACEIs)) became available, placebo-controlled trials documented that these agents may reduce the incidence of adverse events in hypertensive individuals, while providing equivalent efficacy in reducing blood pressure. However, none of these trials were performed and completed using large populations of high-risk patients, and as such, limited conclusions could be drawn with respect to patients with hypertension and CAD.

Current trends in hypertension management emphasize multidrug regimens rather than monotherapy. Combinations of antihypertensive drugs with complementary actions may minimize adverse effects and reduce clinical outcomes by improving blood pressure control and organ protection. β-Blockers are effective in hypertension treatment and reduce incidence of death and reinfarction in patients who have had a myocardial infarction (MI). Along with diuretics, β-blockers became the standard of care for hypertensive CAD patients. However, β-blockers may be less effective antihypertensive agents in older patients, who are also more likely to have CAD. The possibility that other antihypertensive regimens, particularly those containing calcium antagonists and/or angiotensin II active agents, might be as or snore effective than β-blocker and/or diuretic regimens has not been convincingly demonstrated. Previous trials were performed predominantly in populations with low frequencies of CAD and used dihydropyridine calcium antagonists. A recent trial in high-risk hypertensive patients showed that a combination of an angiotensin II-receptor blocker and a diuretic was more effective than a combination of a β-blocker and a diuretic.

Heart rate-reducing nondihydropyridine calcium antagonists, on the other hand, have rarely been studied in large randomized hypertension trials, although verapamil appears to reduce the risk of death and reinfarction in acute CAD trials. The combination of a nondihydropyridine calcium antagonist and an angiotensin-converting enzyme (ACE) inhibitor may provide better blood pressure control and organ protection than monotherapies. Many recent trials indicate that drugs influencing the actions of angiotensin II can be beneficial in high-risk patients, but no hypertension trial has prospectively used these agents for CAD patients with diabetes, renal impairment, or heart failure.

As such, a better understanding of how these newer anti-hypertensive agents may be used to treat hypertension is needed, particularly in patients with CAD, diabetes, or those at risk for strokes. Also, a better understanding of how these newer anti-hypertensive agents may be used in combination is needed. Accordingly, we designed a randomized trial, the INternational VErapamil-trandolapril STudy (INVEST), to determine the effects of a CA BP treatment strategy (CAS) versus a non-CA strategy (NCAS) in hypertensive CAD patients. In particular, INVEST determined the effects when verapamil, (a non-DHP CA), versus atenolol (a beta-adrenergic blocker), was used in combination with an ACEI, such as trandolapril, and a diuretic, such as hydrochlorothiazide (HCTZ).the International Verapamil-Trandolapril Study (INVEST), to compare outcomes in older hypertensive patients with CAD treated with a calcium antagonist strategy (CAS; verapamil sustained release [SR]) or a non-calcium antagonist strategy (NCAS; atenolol). Because most older hypertensive patients require more than 1 agent to adequately control blood pressure, INVEST was intended to compare multidrug strategies rather than individual agents.

### SUMMARY

Described herein is a pharmaceutical composition comprising a combination of anti-hypertensive agents, such as angiotensin-converting-enzyme inhibitors (ACEIs), calcium antagonists (CAs) (preferably non-dihydropyridine (non-DHP) calcium antagonists), and diuretics. Preferably, the ACEI is trandolapril; the CA is verapamil; and the diuretic is hydrochlorothiazide (HCTZ). Derivatives of verapamil, trandolapril, and HCTZ, with similar therapeutic activities may be used as well. The pharmaceutical composition also typically contains one or more pharmaceutical carriers or excipients.

In one embodiment of the pharmaceutical composition, trandolapril, or a derivative thereof, may be present in any amount (e.g., between about 0.5 mg and about 8 mg); verapamil, or a derivative thereof, may be present in any amount (e.g., between about 40 mg and about 480 mg); HCTZ, or a derivative thereof, may be present in any amount (e.g., between about 6.25 mg and about 100 mg); with the proviso that if the composition comprises 4 mg trandolapril or 8 mg trandolapril and the composition comprises 12.5 mg hydrochlorothiazide or 25 mg hydrochlorothiazide, the composition does not comprise 180 mg verapamil; and with the proviso that if the composition comprises 4 mg trandolapril and 12.5 mg hydrochlorothiazide, the composition does not comprise 360 mg verapamil. Preferred amounts of trandolapril are about 0.5 mg, 1 mg, 2 mg, 4 mg, 6 mg, and 8 mg; preferred amounts of verapamil are about 40 mg, 80 mg, 100 mg, 120 mg, 180 mg, 200 mg, 220 mg, 240 mg, 300 mg, 360 mg, and 480 mg; and preferred amounts of HCTZ are about 6.25 mg, 12.5 mg, 25 mg, 37.5 mg, 50 mg, 75 mg, and 100 mg.

In the aforementioned pharmaceutical compositions, one or more of trandolapril, verapamil, HCTZ, and/or their derivatives may function as active ingredients to achieve a therapeutic result. For example, trandolapril verapamil, and/or hydrochlorothiazide may function as anti-hypertensive agents to treat hypertension and to achieve and maintain target blood pressures. As such, trandolapril, verapamil, and/or hydrochlorothiazide may be present in an effective amount to achieve and maintain target blood pressures when the compositions are administered to a patient (e.g., a patient with CAD). A preferable target blood pressure may include a systolic blood pressure (SBP) of no more than about 150, 145, or 140 mm Hg (or 150-140 mm Hg) and a diastolic blood pressure (DBP) of no more than about 90 mm Hg. More preferably, a target blood pressure may include a systolic blood pressure (SBP) of no more than about 140 mm Hg and a diastolic blood pressure (DBP) of no more than about 90 mm Hg. In other embodiments, the aforementioned compositions may be administered to a patient to achieve and maintain other target blood pressures (e.g., a systolic blood pressure of no more than about 135 mm Hg and a diastolic blood pressure of no more than about 85 mm Hg, or alternatively, a systolic blood pressure of no more than about 130 mm Hg and a diastolic blood pressure of no more than about 80 mm Hg). As such, the active ingredients may be useful for decreasing SBP and DBP (e.g., by about 10% or more relative to a baseline measurement.) The above-described target blood pressures may represent mean blood pressures as determined over a set time period.

The aforementioned pharmaceutical compositions may be formulated for controlled release. For example, the pharmaceutical compositions may be formulated together with a matrix that delays the release of one or more of the active ingredients. As such, in one embodiment, the aforementioned compositions may provide effective blood pressure control or other therapeutic effects for about 24-30 hours when administered to a patient. In another embodiment, the aforementioned compositions provide effective blood pressure control or other therapeutic effects for days, weeks (e.g., about 7 days), and/or months (e.g., about 30 days) when administered to a patient. “Effective blood pressure control” may mean achieving and maintaining target blood pressures as described above, (e.g., SBP of no more than about 150, 145, 140, 135, or 130 mm Hg, and DBP of no more than about 90, 85, or 80 mm Hg). “Effective blood pressure control” may also mean a decrease in SBP and DBP (e.g., by at least about 10% relative to baseline measurements).

The aforementioned pharmaceutical compositions may be formulated for any suitable method of administration (e.g., oral, topical, transdermal, subcutaneous, parenteral, or pulmonary administration, and preferably oral, transdermal, or parenteral administration). Oral and/or other formulations may include tablets, capsules, granules, powders, suspensions, liquids, and/or emulsions.

Also disclosed herein is a method of treating hypertension in a patient to achieve and maintain a target blood pressure by administering the above-described compositions. A preferable target blood pressure may include a SBP of no more than about 150, 145, or 140 mm Hg and a DPB of no more than about 90 mm Hg; more preferably a SBP of no more than about 135 mm Hg and DBP of no more than about 85 mm Hg; and even more preferably a SBP of no more than about 130 mm Hg and a DBP of no more than about 80 mm Hg. The method may also be used to decrease SBP and DBP relative to baseline measurements (e.g., by at least about 10%).

The method includes administering a composition that comprises one or more pharmaceutically suitable carriers or excipients and active ingredients including an angiotensin-converting-enzyme inhibitor, a calcium channel blocker (preferably a non-DHP CA), and a diuretic. One or more of the active ingredients are present in an effective amount to treat hypertension. Any of the aforementioned pharmaceutical compositions may be administered in the method. Preferably, the angiotensin-converting-enzyme inhibitor is trandolapril; the calcium channel blocker is verapamil; and the diuretic is HCTZ. Derivatives of trandolapril, verapamil, and HCTZ with similar therapeutic activities may be used as well.

The method may include administering any of the aforementioned compositions to a patient, for whom anti-hypertensive treatment is needed or desirable. In certain embodiments, the patient may have or be at risk for acquiring one or more of coronary artery disease (CAD), renal disease, or diabetes. As such, it may be desirable to administer the aforementioned compositions to treat these diseases or to decrease the likelihood of a patient acquiring these diseases.

In addition to achieving and maintaining a target blood pressure, the method may also be used to decrease the likelihood of a heart attack, and/or to decrease the likelihood of a stroke. Further, the method may be useful in decreasing mortality (e.g., in patients with CAD).

It also may be desirable to administer particular pharmaceutical compositions to a patient based on the patient's age (e.g., an elderly patient of more than about 60 or about 70 years of age), or the patient's ancestry (e.g., African or Hispanic ancestry). A patient's age or ancestry may influence how the patient will responds to particular amounts of verapamil, tranolapril, and HCTZ, and as such, the pharmaceutical composition may be formulated accordingly.

Also disclosed is a kit for treating hypertension in a patient. The kit includes an angiotensin-converting-enzyme inhibitor (preferably trandolapril or a therapeutic derivative), a calcium antagonist (preferably a non-DHP CA such as verapamil or a therapeutic derivative), and a diuretic, (preferably HCTZ or a therapeutic derivative), one or more of which acts as an active ingredient to treat, prevent, and/or ameliorate hypertension. The kit also typically includes one or more pharmaceutically acceptable excipients. The kit includes one or more dosage units, (e.g., up to 35 dosage units), which units may include one or more of trandolapril, verapamil, HCTZ, or their therapeutic derivatives. The dosage units may be formulated as a pharmaceutical composition that includes the one or more pharmaceutically suitable carriers or excipients.

The kit may include trandolapril, verapamil, HCTZ, or their therapeutic derivatives, in any of the amounts described in the aforementioned pharmaceutical compositions as active ingredients. The active ingredients may be formulated separately in the kit (i.e., separate first, second, and third dosage units including trandolapril, verapamil, and hydrochlorothiazide, respectively), or they may be formulated in any combination as dosage units (e.g., a dosage unit including trandolapril, verapamil, and HCTZ or a dosage unit including trandolapril and verapamil). The dosage unit(s) may include an effective amount of one or more of the active ingredients to treat hypertension.

The kit also includes instructions for use to treat, prevent and/or ameliorate hypertension in a patient in need thereof. For example, the instructions for use may indicate how to treat, prevent and/or ameliorate hypertension in a patient in need thereof with regard to the patient's current condition, (e.g., in a patient with one or more of hypertension and/or CAD, renal disease, and diabetes, or at risk for acquiring these diseases.) The instructions for use may also indicate how to treat, prevent and/or ameliorate hypertension in a patient in need thereof with regard to the patient's age or ancestry. The instructions for use may also include recommended dosage amounts and method of administration to achieve and maintain target blood pressures (e.g., a SBP of no more than about 150, 145, 140, 135, or 130 mm Hg and a DBP of no more than about 90, 85, or 80 mm Hg in the patient). The kit also may include instructions for reducing SBP and DBP relative to baseline measurements (e.g., by at least about 10%). The kit also may include one or more implements to facilitate administering the dosage units.

### BRIEF DESCRIPTION OF THE DRAWINGS

FIG. 1 is a schematic representation of the INVEST trial profile and study protocol.

FIG. 2 displays the mean systolic and diastolic blood pressure during the trial.

FIG. 3 displays the primary outcome of the trial by treatment strategy.

FIG. 4 displays the primary and secondary outcomes of the trial by treatment strategy.

FIG. 5 displays the effects of treatment strategy on primary outcome in subgroups of patients at baseline.

### DETAILED DESCRIPTION

Unless otherwise specified, “a” or “an” means one or more.

Described herein is a pharmaceutical composition comprising a combination of anti-hypertensive agents, such as angiotensin-converting-enzyme inhibitors (ACEIs), calcium antagonists (CAs), and diuretics. Preferably, the ACEI is trandolapril; the CA is a non-DHP CA (e.g., verapamil); and the diuretic is hydrochlorothiazide (HCTZ). Therapeutic derivatives of trandolapril, verapamil, and HCTZ may be used as well.

The term “therapeutic derivative” is intended to mean any compound that has a related structure and achieves a similar therapeutic result. “Therapeutic derivatives” may include pharmaceutically acceptable or suitable salts. “Therapeutic derivatives” may also include compounds that differ structurally from trandolapril, verapamil, and HCTZ, prior to being administered to a patient, but which are converted to therapeutically similar structures after being administered to a patient, (e.g., an oral, acid-activated prodrug of trandolapril, verapamil, or HCTZ that is hydrolyzed in the stomach to produce the active drug form.) Angiotensin-converting-enzyme inhibitors, such as trandolapril or therapeutic derivatives thereof, and/or combinations of ACEI's with calcium antagonists or diuretics (e.g., TARKA), are described in U.S. Pat. No. 4,933,361; U.S. Pat. No. 5,098,910; U.S. Pat. No. 5,403,856; U.S. Pat. No. 5,500,434; U.S. Pat. No. 5,684,016; U.S. Pat. No. 5,721,244; U.S. Pat. No. 5,744,496; and U.S. Pat. No. 5,747,504, which are incorporated herein by reference in their entireties.

The pharmaceutical composition also typically contains a pharmaceutical suitable carrier or excipient, which is intended to mean substances, which are substantially harmless to the individual to which the dosage unit will be administered. Such an excipient normally fulfills the requirements given by national drug agencies. Official pharmacopeias such as the U.S.A. Pharmacopeia, the British Pharmacopeia, and the European Pharmacopeia set standards for well-known pharmaceutically acceptable carriers and excipients.

Suitable carriers and excipients may include all kinds that may be used for solid, semi-solid, fluid, or other dosage units. Suitable carriers and excipients may include solvents, buffering agents, preservatives, humectants, chelating agents, antioxidants, stabilizers, emulsifying agents suspending agents, gel-forming agents, diluents, disintegrating agents, binding agents, lubricants, coating agents, and wetting agents. Typically, the diluents and disintegrating agents may be lactose, saccharose, calcium phosphatases, calcium carbonate, calcium sulfate, mannitol, starches, and cellulose.

Binding agents may include saccharose, sorbitol, gum acacia, sodium alginate, gelatin, starches, cellulose, sodium carboxymethylcellulose, methylcellulose, hydroxypropylcellulose, polyvinylpyrrolidone, and polyethyleneglycol.

Wetting agents may include by example sodium laurylsulfate and polysorbate 80. Lubricants may include by example talcum, magnesium stearate, calcium stearate, silicium oxide, and polyethyleneglycol. Coating agents may include by example hydroxypropylcellulose, hydroxypropylmethylcellulose, polyvinylpropylidone, ethylcellulose, and polymethylacrylates.

In one embodiment of the pharmaceutical composition, trandolapril, or a derivative with a similar therapeutic effect, may be present in any amount (e.g., between about 0.5 mg and about 8 mg or preferably between about 0.5 mg to about 2 mg); verapamil, or a derivative with a similar therapeutic effect, may be present in any amount (e.g., between about 40 mg and about 480 mg or preferably about 180 to about 240 mg); and HCTZ, or a derivative with a similar therapeutic effect, may be present in any amount (e.g., between about 6.25 mg and about 100 mg or preferably between about 6.25 mg and 25 mg); with the proviso that if the composition comprises 4 mg trandolapril or 8 mg trandolapril and the composition comprises 12.5 mg hydrochlorothiazide or 25 mg hydrochlorothiazide, the composition does not comprise 180 mg verapamil; and with the proviso that if the composition comprises 4 mg trandolapril and 12.5 mg hydrochlorothiazide, the composition does not comprise 360 mg verapamil.

In other embodiments, the pharmaceutical composition may include particular amounts of trandolapril, verapamil, HCTZ, or their therapeutic derivatives. Non-limiting examples include: (trandolapril: about 0.5, 1, or 2 mg; verapamil: about 40, 80, 100, 120, 180, 200, 220, 240, 360, or 480 mg; HCTZ about 6.25, 12.5, 25, 50, 75, or 100 mg), (trandolapril: about 1 or 2 mg; verapamil: about 180 or 240 mg; HCTZ: about 6.25, 12.5, 25, 50, 75, or 100 mg), (trandolapril: about 2 mg; verapamil: about 180 mg; HCTZ: about 6.25, 12.5, 25, 50, 75 or 100 mg), (trandolapril: about 2 mg; verapamil: about 180 mg; HCTZ about 12.5, 25, or 50 mg), (trandolapril: about 2 mg; verapamil: about 180 mg; HCTZ about 12.5 or 25 mg). As used herein, “about” means +/−20%, preferably +/−10%, and more preferably +/−5%.

In the aforementioned pharmaceutical compositions, one or more of trandolapril, verapamil, HCTZ, and/or their therapeutic derivatives may function as active ingredients that exhibit an anti-hypertensive effect in a patient (e.g., a patient with CAD). An anti-hypertensive effect may be observed by taking a sitting cuff blood pressure measurement of systolic and diastolic blood pressure before and after the pharmaceutical composition is administered. In one preferable embodiment, one or more of trandolapril, verapamil, and/or hydrochlorothiazide may be present as active ingredients in a dose sufficient to achieve and maintain a SBP of no more than about 150-140 mm Hg and a DBP of no more than about 90 mm Hg; more preferably a SBP of no more than about 135 mm Hg and a DBP of no more than about 85 mm Hg, and even more preferably SBP of no more than about 130 mm Hg and a DBP of no more than about 80 mm Hg. The active ingredients may also be present in an effective amount to reduce SBP and DBP relative to baseline measurements taken prior to administering the composition. Preferably, the active ingredients are present in an effective amount to reduce SBP and DBP by at least about 10% relative to baseline measurements. The above-described target blood pressures may represent mean blood pressures as determined over a set time period.

The aforementioned compositions may be formulated for oral, topical, transdermal, subcutaneous, parenteral, or pulmonary (e.g., aerosolized) administration. Oral, transdermal, and/or parental formulations are preferable. Oral and/or other formulations may include tablets, capsules, granules, powders, suspensions, liquids, and/or emulsions. Transdermal formulations may include patches or pads.

The aforementioned pharmaceutical compositions may be formulated together with a matrix that controls the release of one or more of the active ingredients (e.g., a matrix for slow release of the active ingredients). The matrix typically is a solid formulation which allows for the controlled, prolonged, or extended release of an active ingredient at a rate sufficient to maintain therapeutic blood levels of the active ingredient over a period of time (e.g., 24-30 hours, 1-7 days, 1-30 days or longer). The matrix can represent from about 40% to about 98% of the total weight of a pharmaceutical composition or a unit dosage form, typically excluding any coatings in the case of tablets. More preferably the controlled release matrix will represent from about 50% to about 95% of the total weight of the inventive compositions. The matrix to active ingredient ratio can be from about 5 to 1 to about 15 to 1, and compositions having integer ratios of all possible combinations between these ranges including 10 to 1 are considered embodiments of the present invention.

The matrix can be any suitable material that provides sustained, controlled, or slow release of an active ingredient, medicament or drug and the like. Pharmaceutically acceptable rate controlling materials which may be used in the present invention include both synthetic and naturally occurring gums and/or polymers and other art-known rate controlling substances. Non-limiting examples include naturally occurring or modified naturally occurring or synthetic or semi-synthetic polymers or gums such as, e.g., alginates, carrageenan, pectin, xanthan gum, locust bean gum, guar gum, modified starch, alkylcellulose, hydroxypropylmethylcellulose, methylcellulose, and other cellulosic materials or polymers, such as sodium carboxymethylcellulose and hydroxypropylcellulose and mixtures of the foregoing. Additional synthetic and/or semisynthetic polymers include, e.g., cellulose acetate phthalate (CAP), polyvinylacetate phthalate (PVAP), hydroxypropylmethylcellulose phthalate, and/or acrylic polymers, such as methacrylic acid ester copolymers, zein, and the like. The matrix can include ingredients such as polysaccharides, cationic crosslinking agents, inert diluents, alkalizing agents, surfactants, polar solvents and other excipients.

The controlled release matrix may be formulated to provide effective blood pressure control or other therapeutic effects for hours (e.g., 24-30 hours), days (e.g., up to 7), weeks (e.g., up to 4), and/or months (e.g., up to 12), by maintaining an effective concentration of one or more of the active ingredients (e.g., trandolapril, verapamil, and/or HCTZ) in the patient's sera. “Effective blood pressure control,” as defined herein, may mean a SBP of no more than about 150, 145, 140, (i.e., 150-140), 135, and 130 mm Hg and a DBP of no more than about 90, 85, or 80 mm Hg. “Effective blood pressure control,” as defined herein, may also mean a decrease in SBP and DBP by at least about 10% relative to baseline measurements. “Baseline measurements,” as defined herein, may mean SBP and DBP measurements taken before the composition is administered.

The above-described pharmaceutical compositions may be administered to a patient to achieve and maintain a target blood pressure. Methods of administration are well known in the art and may vary based on the particular formulation, (i.e., oral, transdermal, parenteral, etc.). Methods of administration may also vary based on whether the pharmaceutical composition is formulated for sustained, controlled, or slow release (e.g., by using a controlled release matrix).

The method may include administering any of the aforementioned compositions to any patient, for whom anti-hypertensive treatment is needed or desirable. The method may be particularly beneficial for patients who may have coronary artery disease. “Coronary artery disease” or “CAD,” as defined herein, means remote (≧3 months prior) confirmed MI, coronary angiogram with more than 50% narrowing of at least 1 major coronary artery, diagnosis of classic angina pectoris, or concordant abnormalities on 2 different types of signals (electrocardiograms, echocardiograms, and/or radionuclide scans) from stress tests provided that 2 different signals showed findings consistent for ischemia (e.g., ST-segment depression and/or perfusion defects by radionuclide, and/or wall-motion abnormalities by echocardiogram or radionuclide). The method may also be beneficial for patients with renal disease, or patients with diabetes or at risk for acquiring diabetes. For example, the method may be useful to reduce blood pressure and to decrease the likelihood of a patient acquiring diabetes or to delay the onset of diabetes in a patient. In addition to reducing blood pressure, the method may be beneficial also in reducing mortality, likelihood of a heart attack (and/or a myocardial infarction), renal failure, and/or stroke.

It may be desirable to administer particular compositions to a patient based on the patient's age (e.g., an elderly patient older than about 70 years or older than about 60 years), or ancestry, such as African or Hispanic ancestry. For example, because hypertensive patients of African ancestry (i.e., black hypertensives) may respond differently to certain anti-hypertensive agents in comparison to Caucasian hypertensives, it may be desirable to administer different compositions to black hypertensives to achieve target blood pressures.

The kit for treating, preventing, and/or ameliorating hypertension in a patient (e.g., a patient with CAD) includes an angiotensin-converting-enzyme inhibitor, a calcium antagonist (preferably a non-DHP CA), and a diuretic. Typically, trandolapril, verapamil, HCTZ, or their therapeutic derivatives, as active ingredients, are present in the kit in any of the amounts described in the aforementioned pharmaceutical composition (i.e., trandolapril: 0.5-8 mg; verapamil: 40-480 mg; and HCTZ: 6.25-100 mg). One or more of the active ingredients is present in an effective amount to treat hypertension. Trandolapril, verapamil, HCTZ, and/or their therapeutic derivatives may be formulated separately or in combination as dosage units (e.g., trandolapril and verapamil may be formulated in combination as a dosage unit and/or trandolapril, verapamil, and HCTZ may be formulated in combination as a dosage unit). As such, a dosage unit may include one or more of trandolapril, verapamil, and/or HCTZ, one or more of which acts as an active ingredient to treat, prevent, and/or ameliorate hypertension.

The kit includes one or more dosage units, and it may be desirable to create kits that contain up to 35 dosage units (e.g., where a dosage unit is to be administered daily and the kit is designed to last for up to one month).

The kit also typically includes one or more pharmaceutically suitable carriers or excipients, as described above, which may be present in a dosage unit (e.g., the carrier and/or excipient may be formulated together with one or more active ingredient in a dosage unit.)

The kit may be used to treat or decrease the likelihood of coronary artery disease, renal failure, stroke, or diabetes. The kit may also be used to delay the onset of diabetes, and/or to decrease mortality (e.g., as a result of treating any of the aforementioned diseases or conditions).

The kit includes instructions for using the kit for treatment, prevention or amelioration of hypertension. As used herein, the phrase “instructions for use” or “instructions for using” shall mean any FDA-mandated instructions, package inserts, or labels that relate to the administration of the aforementioned compositions or the dosage units in the kit for the purpose of treating hypertension, or the equivalent instructions, package inserts, or labels required by foreign regulatory authorities. For example, instructions for use may include, but are not limited to, indications for hypertension, identification of specific symptoms of hypertension that can be ameliorated by the aforementioned compositions or dosage units, and recommended dosage amounts for patients suffering from hypertension. The instructions for use may indicate that the kit may be used to treat or decrease the likelihood of acquiring coronary artery disease, to decrease mortality, to decrease the likelihood of a heart attack and/or myocardial infarction, and/or to decrease the likelihood of a stroke. The instructions for use may indicate particular dosages for using the kit to treat, prevent, and/or ameliorate hypertension by achieving and maintaining target blood pressures (e.g., a SBP of no more than about 150, 145, 140, 135, Or 130 mm Hg and a DBP of no more than about 90, 85, Or 80 mm Hg). The instruction may also indicate how to decrease SBP and DBP relative to baseline measurements (e.g., by at least about 10%).

### EXAMPLES

The INVEST design and methods have been described in detail elsewhere. (See, e.g., Pepine C J, et al., J Am Coll Cardiol 1998; Vol. 32: 1228-37 and Pepine et al., JAMA (2003), Vol. 290:2805-2816 and E1-E3, incorporated herein by reference in their entireties). INVEST was an international, multicenter study with a prospective, randomized, open blinded end-point evaluation design conducted according to principles of the Declaration of Helsinki. The institutional review boards and ethics committees at participating sites approved the protocol and patients provided written informed consent.

INVEST Design and Patients

Clinically stable CAD patients with hypertension were randomly assigned to either a CAS or NCAS for BP treatment at 862 sites in 14 countries and managed according to the Sixth Report of the Joint National Committee on Prevention, Detection, Evaluation, and Treatment of High Blood Pressure (JNC VI) guidelines. (Arch Intern Med 1997; Vol. 157: 2413-46 [Erratum, Arch Intern Med 1998; Vol 158: 573], incorporated herein by reference in their entireties). The primary objective was to evaluate the hypothesis that risk for adverse outcomes is at least equivalent during treatment initiated with a CAS compared with an NCAS. The primary outcome was the first occurrence of death (all-cause), nonfatal MI, or nonfatal stroke.

One objective of the study was to test the hypothesis that risk for adverse outcomes is equivalent to a verapamil SR-based regimen compared with an atenolol-based regimen. Clinically stable CAD patients with hypertension were randomly assigned to either verapamil SR or atenolol for blood pressure treatment according to the sixth report of the Joint National Committee on Prevention, Detection, Evaluation, and Treatment of High Blood Pressure (JNC VI) (target: systolic blood pressure [SBP]<140 and diastolic blood pressure [DBP]<90 mm Hg or SBP<130 mm Hg and DBP<85 mm Hg when diabetes or renal impairment is present). Addition of trandolapril and/or hydrochlorothiazide was recommended when necessary to achieve blood pressure goals. Trandolapril also was recommended for patients with heart failure, diabetes, or renal insufficiency. Thus, this was not simply a comparison of verapamil SR with atenolol because it was anticipated that few patients would be treated with only those drugs. Ultimately, it was expected that most patients would be using the combination of verapamil SR plus trandolapril or atenolol plus hydrochlorothiazide.

Patient were eligible for the study if they were aged 50 years or older and had documented CAD, with essential hypertension as defined by JNC VI requiring drug therapy. Documented CAD was defined as any of the following: remote (≧3 months prior to enrollment) confirmed MI, coronary angiogram with more than 50% narrowing of at least 1 major coronary artery, diagnosis of classic angina pectoris, or concordant abnormalities on 2 different types of signals (electrocardiograms, echocardiograms, and/or radionuclide scans) from stress tests provided that 2 different signals showed findings consistent for ischemia (eg, ST-segment depression and/or perfusion defects by radionuclide, and/or wall-motion abnormalities by echocardiogram or radionuclide). Patients with heart failure classes I through III were included. Patients taking β-blockers within 2 weeks of randomization or taking β-blockers for an MI that occurred in the previous 12 months were excluded to avoid withdrawal phenomena in patients randomized to the CAS group.

Following validity checks of eligibility data, an Internet-based management system automatically randomized each patient to a treatment strategy. The randomization scheme used a standard C routine and blocked by site using randomly permuted block sizes of 4 and 6. The randomization result was automatically stored in the central database as part of the patient's record and was also returned to the site investigator for electronic signature of strategy drugs in accordance with the protocol.

Interventions

The protocol-recommended treatment schedule for each strategy to achieve JNC VI blood pressure targets is outlined as follows. The blood pressure target was determined from a mean of 2 sitting cuff blood pressure measurements as described in JNC VI.

Patients allocated to the CAS group were given 240 mg/d of verapamil SR while patients allocated to the NCAS group were given 50 mg/d of atenolol (step 1). If patients did not achieve target blood pressure, in step 2 the CAS group also could receive trandolapril (an ACE inhibitor) and the NCAS group also could receive hydrochlorothiazide. The rationale for this was to maximize use of the combination of calcium antagonist and ACE inhibitor while minimizing diuretic use for the CAS group and maximizing use of the combination of β-blocker and diuretic for NCAS group. In step 3, doses were increased in both groups. In step 4, the CAS group also could receive hydrochlorothiazide and the NCAS group also could receive trandolapril. Trandolapril was recommended for all patients with renal impairment, diabetes, or heart failure. If the dose was not well tolerated or the target blood pressure was not achieved, verapamil SR could be titrated to between 120 and 480 mg/d and atenolol could be titrated to between 25 and 200 mg/d. The recommended starting dose for trandolapril was 2 mg/d and it could be titrated to between 0.5 and 8 mg/d. For patients in the CAS group, a fixed combination was available for verapamil SR and trandolapril in doses of 180 mg/d and 2 mg/d, respectively; 240 mg/d and 1 mg/d; and 240 mg/d and 4 mg/d. The recommended starting dose for hydrochlorothiazide was 25 mg/d and it could be titrated between 12.5 and 100 mg/d. Doses greater than 25 mg of hydrochlorothiazide were provided to limit the need for nonstudy diuretics in patients with heart failure or edema. If the blood pressure goal was not achieved and adverse effects had not occurred, doses were titrated to those levels as specified in the instant step before a patient was moved to the next step.

Additional non-study antihypertensive drugs, (except β-blockers for CAS patients and calcium antagonists for NCAS patients), could be added when needed to reach blood pressure targets or minimize adverse effects. Patients were considered to have crossed over from their randomized treatment strategy if they received a β-blocker during the trial and were in the CAS group or received a calcium antagonist and were in the NCAS group. Standard of care, non-pharmacological JNC VI guidelines, and secondary prevention according to the National Cholesterol Education Program were provided online to physicians, which could be printed and given to patients.

Patient Monitoring and Follow-Up

Protocol visits were scheduled every 6 weeks for the first 6 months and then biannually until 2 years after the last patient was enrolled. Patients were assessed for response to treatment, occurrence of symptoms, treatment compliance, and adverse effects at each visit and at study close as detailed elsewhere.

Patient follow-up was complete when a final assessment form was received via the online data system or a death report was received. For all patients not completing the final assessment visit, lost to follow-up, or withdrawn, data were censored according to last visit date.

Study Outcomes

The primary outcome was the first occurrence of death (all-cause), nonfatal MI, or nonfatal stroke by intention-to-treat analysis. The MI and stroke definitions are detailed on the INVEST Web site. These 3 components individually were the main secondary outcomes. Additional outcomes included time to most serious event (ranked from death as most serious, to MI, to stroke as least serious), cardiovascular death (definite or presumed), angina, cardiovascular hospitalizations, blood pressure control, cancer, Alzheimer disease, Parkinson disease, and gastrointestinal tract bleeding. Shortly after the study started, new information became available on the potential for ACE inhibitors to prevent or delay the onset of diabetes. Accordingly, at the recommendation of the independent data safety and monitoring committee, new diagnosis of diabetes was added as an outcome early in the recruitment phase of the study.

Outcomes such as death, MI, stroke, and cardiovascular hospitalization were reported within 24 hours using the online adverse event reporting system and then appropriate documentation was gathered. Adverse experiences were collected from responses to open, active questioning not restricted to those events known to be associated with the drugs taken. Three members of the events committee, masked to treatment assignment, confirmed all outcome events by reviewing documentation and other pertinent patient records. The data safety and monitoring committee reviewed efficacy and safety data at regular intervals throughout the trial.

Sample Size

It was decided a priori that a 20% difference in primary outcome between the treatment strategies would be clinically relevant using the intention-to-treat population. Therefore, the equivalence bound for the risk ratio was a confidence interval (CI) of 1.20 to 0.83. We assumed an annual primary outcome rate of no less than 2%, an α of 0.05 (2-sided), and 90% power when estimating the number of patients required. On this basis, a tentative sample size of 27000 patients was calculated, with an anticipated yearly drop-out rate of 5% to 10%. Because the enrollment period was longer than initially planned, patient-years of follow-up were greater than those used for initial power estimates. At the recommendation of the INVEST study biostatisticians and the data safety and monitoring committee, the steering committee reduced the sample size to 22000 patients.

Statistical Analysis

All of the main analyses were completed as specified in the protocol with the intention-to-treat population, including patients withdrawn or lost to follow-up censored at the time of the last visit (unless the patient was known to be dead based on death records). One planned interim analysis was performed in August 2001 and the pre-specified stopping rules were not met.

The final significance level for the primary outcome, adjusted for the single interim analysis, was P=0.04806 for a 2-sided test. For the secondary outcomes of death, nonfatal MI, and nonfatal stroke, a Bonferroni adjustment was made to the same P=0.04806 significance level (P=0.02 for each outcome). All other analyses are reported at the P<0.05 significance level. Kaplan-Meier survival analysis was used to assess time to first event for the primary outcome and the main secondary outcomes. The primary outcome was analyzed both unadjusted and adjusted for 5 pre-specified covariates: age, race, sex, previous MI, and prior heart failure. Standard relative risk (RR) estimates and 95% CIs were also calculated.

χ² Analysis was used to compare CAS with NCAS on percentage occurrence of different outcomes. Cox proportional hazard models were used to evaluate potential interactions in the reported pre-specified subgroup analyses (by baseline characteristic). All data were captured and stored in database tables (Version 7.1, Oracle, Redwood Shores, Calif). Data management and statistical analyses were performed using SAS statistical software (Version 8.2, SAS Institute Inc, Cary, N.C.). The database was maintained at the University of Florida, Division of Biostatistics, Gainesville.

Patient Enrollment

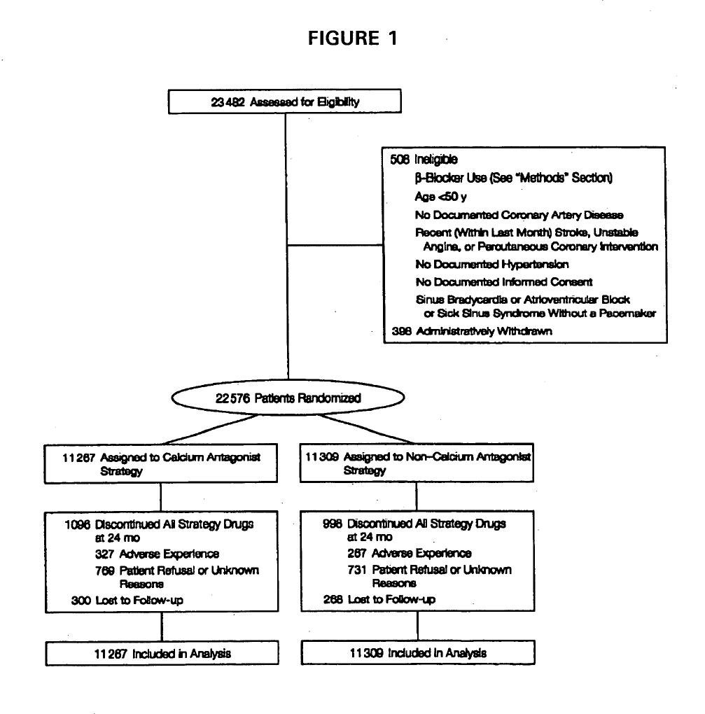

The pilot phase (30 selected sites) started in September 1997. Full-scale site recruitment and patient enrollment began in January 1998, and patient follow-up was completed on Feb. 14, 2003. A total of 22576 patients at 862 sites in 14 countries provided informed consent, satisfied administrative requirements, and completed randomization; 11267 were assigned to the CAS group and 11309 to the NCAS group (FIG. 1). A total of 594 patients had all assigned drugs withdrawn due to an adverse experience. A total of 568 patients failed to return for final assessment and did not appear in death searches (withdrawals or lost to follow-up). These latter patients were censored at the time of their last visit. Mean follow-up was 2.7 years (range, 1 day to 5.4 years) in each strategy. A total of 30829 patient-years were accumulated in the CAS group and 31006 patient-years in the NCAS group.

Baseline Characteristics

At baseline, patient characteristics were well-balanced (TABLE 1). The study population included a large proportion of elderly, Hispanic, diabetic, and female patients. Blood pressure levels were similar between groups (TABLE 2). Overall, only 4267 patients (18.9% of all patients) had controlled blood pressure.

TABLE 1
Patient Characteristics at Baseline*

| | | |
|---|---|---|
| | Calcium | Non-Calcium |
| | Antagonist | Antagonist |
| | Strategy | Strategy |
| Characteristic | (n = 11 267) | (n = 11 309) |

Demographic

| | | |
|---|---|---|
| Age, mean (SD), y | 66.0 (9.7) | 66.1 (9.8) |
| >70 | 3694 (32.8) | 3829 (33.9) |
| Women | 5850 (51.9) | 5920 (52.3) |
| Race/ethnicity | | |
| White | 5466 (48.5) | 5459 (48.3) |
| Black | 1506 (13.4) | 1523 (13.5) |
| Hispanic | 4021 (35.7) | 4024 (35.6) |
| Asian | 63 (0.6) | 86 (0.8) |
| Other/multiracial | 211 (1.9) | 217 (1.9) |
| BMI, mean (SD), kg/m² | 29.1 (6.8) | 29.2 (7.4) |

Condition

| | | |
|---|---|---|
| Myocardial infarction | 3622 (32.1) | 3596 (31.8) |
| Abnormal angiogram | 4384 (38.9) | 4472 (39.5) |
| Prior MI or abnormal angiogram | 5932 (52.6) | 6025 (53.3) |
| Concordant stress test | 2399 (21.3) | 2389 (21.1) |
| abnormalities | | |
| Angina pectoris | 7463 (66.2) | 7582 (67.0) |
| CABG ≧1 month ago | 1751 (15.5) | 1821 (16.1) |
| PCI ≧1 month ago | 1716 (15.2) | 1666 (14.7) |
| CABG or PCI | 3079 (27.3) | 3087 (27.3) |
| Stroke | 595 (5.3) | 567 (5.0) |
| Left ventricular hypertrophy | 2422 (21.5) | 2526 (22.3) |
| Unstable angina ≧1 mo ago | 1280 (11.4) | 1298 (11.5) |
| Arrhythmia | 802 (7.1) | 798 (7.1) |
| Heart failure (class I-III) | 619 (5.5) | 637 (5.6) |
| Peripheral vascular disease | 1345 (11.9) | 1354 (12.0) |
| Smoking | | |
| Past | 5247 (46.6) | 5207 (46.0) |
| Within last 30 d | 1435 (12.7) | 1374 (12.2) |
| Never | 6020 (53.4) | 6102 (54.0) |
| Diabetes† | 3169 (28.1) | 3231 (28.6) |
| Hypercholesterolemia† | 6300 (55.9) | 6293 (55.6) |
| Renal impairment‡ | 214 (1.9) | 210 (1.9) |
| Cancer§ | 389 (3.5) | 371 (3.3) |

Medication

| | | |
|---|---|---|
| Aspirin or other antiplatelet agent | 6418 (57.0) | 6377 (56.4) |
| Other NSAIDs | 1984 (17.6) | 2024 (17.9) |
| Antidiabetic medication§ § | 2493 (22.1) | 2591 (22.9) |
| Any lipid-lowering agent | 4150 (36.8) | 4144 (36.6) |
| Nitrates | 3989 (35.4) | 4139 (36.6) |
| Potassium supplement | 777 (6.9) | 783 (6.9) |
| Hormone replacement¶ | 1034 (17.7) | 1096 (18.5) |

Abbreviations:
BMI, body mass index;
CABG, coronary artery bypass graft;
NSAIDs, nonsteroidal anti-inflammatory drugs;
PCI, percutaneous coronary interventions.
*Values expressed as number (percentage) unless otherwise indicated. Percentages may not equal 100 due to rounding.
†History of or currently taking antidiabetic or lipid-lowering medications.
‡History of or currently have elevated serum creatinine level but less than 4 mg/dL (<354 μmol/L).
§Patients with a history of skin, prostate, and other cancers with long survival expectancy were not excluded.
§§ Insulin and/or oral hypoglycemics.
¶Data for women only (n = 5850 for calcium antagonist strategy and n = 5920 for non-calcium antagonist strategy).

TABLE 2
Baseline Blood Pressure and Antihypertensive Medications

| | |
|---|---|
| Calcium | Non-Calcium |
| Antagonist | Antagonist |
| Strategy | Strategy |

Antihypertensive Medication Use

| | (n = 9758) | (n = 9791) |
|---|---|---|
| Blood pressure, mean (SD), mm Hg | | |
| Systolic | 149.5 (19.7) | 149.5 (19.7) |
| Diastolic | 86.3 (12.0) | 86.3 (11.9) |
| Heart rate, mean (SD), beats/min | 75.6 (9.5) | 75.5 (9.5) |
| No. (%) with blood pressure in | | |
| control* | | |
| Systolic | 2384 (24.4) | 2359 (24.1) |
| Diastolic | 5244 (53.7) | 5311 (54.2) |
| Both | 2154 (22.1) | 2113 (21.6) |
| No. of drugs, mean (SD) | 1.7 (0.8) | 1.7 (0.8) |
| 1 | 5030 (51.6) | 4978 (50.8) |
| 2 | 3330 (34.1) | 3355 (34.3) |
| 3 | 1110 (11.4) | 1172 (12.0) |
| >3 | 288 (3.0) | 286 (2.9) |
| Type of antihypertensive | | |
| drug, No. (%) | | |
| ACE inhibitor | 5007 (51.3) | 5042 (51.5) |
| Centrally acting† | 516 (5.3) | 536 (5.5) |
| Calcium antagonist | 4031 (41.3) | 4058 (41.4) |
| Diuretic | 3650 (37.4) | 3743 (38.2) |
| §-Blocker/other vasodilator | 828 (8.5) | 830 (8.5) |
| †-Blocker‡ | 0 | 0 |
| Other class | 2179 (22.3) | 2183 (22.3) |

No Antihypertensive Medication Use

| | (n = 1509) | (n = 1518) |
|---|---|---|
| Blood pressure, mean (SD), mm Hg | | |
| Systolic | 159.2 (15.7) | 160.1 (15.9) |
| Diastolic | 92.9 (10.1) | 92.6 (10.6) |
| Heart rate, mean (SD), beats/min | 76.6 (10.0) | 76.7 (9.7) |

Abbreviations:
ACE, angiotensin-converting enzyme;
CAS, calcium antagonist strategy.
*According to guidelines from the sixth report of the Joint National Committee on Prevention, Detection, Evaluation, and Treatment of High Blood Pressure (see text).
†Included clonidine, methyldopa, and moxonidine.
‡Patients taking †-blockers within 2 weeks of randomization or taking †-blockers for an MI that occurred in the previous 12 months were excluded to avoid withdrawal phenomena in patients randomized to the CAS group.

Treatment

At 24 months, 6391 (81.5%) of CAS patients were taking verapamil SR and 6083 (77.5%) of NCAS patients were taking atenolol (TABLE 3). As expected from the recommended order of additional drug treatment by strategy, usage of trandolapril and hydrochlorothiazide differed significantly (P<0.001). The distribution of number of study drugs used was similar between strategies as was the distribution of total antihypertensive medications. At 24 months, only 2.1% of patients in each group (CAS, 145; NCAS, 141) were taking no antihypertensive medications. At final assessment, nonstudy antihypertensive drug use was observed in 5873 patients (43%) in both strategies (TABLE 4). As expected, calcium antagonist use was more frequent in the CAS group and β-blocker use was more frequent in the NCAS group (TABLE 4). Crossover to β-blocker use in the CAS group (373 [5.5%]) was less than crossover to calcium antagonist use in the NCAS group (479 [7.0%]). This difference persisted over the entire duration of follow-up, β-blocker use at any time in the CAS group was 1305 (11.6%) of 11267 and calcium antagonist use in the NCAS group was 1862 (16.5%) of 11309 (P<0.00I.). Nonstudy diuretic use was also more frequent in the NCAS group. The percentage of patients taking antidiabetic medications was significantly lower in the CAS group (23.2%; n=1574) compared with the NCAS group (24.7%; n=1682) (P=0.04). The frequencies of other medication use were similar between strategies (TABLE 4).

TABLE 3
Strategy Antihypertensive Medication Use
A. No. (%) of Patients at 12 months

| | Calcium | Non-Calcium | |
|---|---|---|---|
| | Antagonist | Antagonist | |
| | Strategy | Strategy | |
| | (n = 8639) | (n = 8694) | P Value* |
| Study drug | | | |
| Verapamil sustained release | 7581 (87.8) | N/A | |
| Mean (SD) dose, mg/d | 274 (99) | N/A | |
| Atenolol | N/A | 7060 (81.2) | |
| Mean (SD) dose, mg/d | N/A | 72 (33) | |
| Trandolapril | 5436 (62.9) | 4514 (51.9) | <.001 |
| Mean (SD) dose, mg/d | 4 (2) | 3 (2) | |
| Hydrochlorothiazide | 3515 (40.7) | 5168 (59.4) | <.001 |
| Mean (SD) dose, mg/d | 28 (14) | 28 (12) | |
| No. of strategy drugs | | | |
| 0 | 715 (8.3) | 708 (8.1) | |
| 1 | 1964 (22.7) | 1920 (22.1) | .73 |
| 2 | 3312 (38.3) | 3376 (38.8) | |
| 3 | 2648 (30.7) | 2690 (30.9) | |
| Total No. of | | | |
| antihypertension drugs | | | |
| (strategy plus nonstrategy) | | | |
| 0 | 117 (1.4) | 126 (1.4) | |
| 1 | 1376 (15.9) | 1294 (14.9) | .18 |
| 2 | 2941 (34.0) | 2931 (33.7) | |
| ≧3 | 4205 (48.7) | 4343 (50.0) | |

B. No. (%) of Patients at 24 months

| | Calcium | Non-Calcium | |
|---|---|---|---|
| | Antagonist | Antagonist | |
| | Strategy | Strategy | |
| | (n = 7842) | (n = 7850) | P Value* |
| Study drug | | | |
| Verapamil sustained release | 6391 (81.5) | N/A | |
| Mean (SD) dose, mg/d | 288 (102) | N/A | |
| Atenolol | N/A | 6083 (77.5) | |
| Mean (SD) dose, mg/d | N/A | 76 (34) | |
| Trandolapril | 4934 (62.9) | 4113 (52.4) | <.001 |
| Mean (SD) dose, mg/d | 4 (2) | 4 (3) | |
| Hydrochlorothiazide | 3430 (47.7) | 4733 (60.3) | <.001 |
| Mean (SD) dose, mg/d | 29 (14) | 29 (13) | |
| No. of strategy drugs | | | |
| 0 | 1096 (14.0) | 998 (12.7) | |
| 1 | 1363 (17.4) | 1424 (18.1) | .11 |
| 2 | 2757 (35.2) | 2779 (35.4) | |
| 3 | 2626 (33.5) | 2649 (33.8) | |
| Total No. of | | | |
| antihypertension drugs | | | |
| (strategy plus nonstrategy) | | | |
| 0 | 145 (2.1)† | 141 (2.1)‡ | |
| 1 | 1061 (15.6)† | 1031 (15.1)‡ | .57 |
| 2 | 2123 (31.3)† | 2089 (30.6)‡ | |
| ≧3 | 3464 (51.0)† | 3561 (52.2)‡ | |

Abbreviation:
N/A, not applicable.
*Results from χ² test to compare strategies.
†Based on reduced sample size of 6793.
‡Based on reduced sample size of 6822.

TABLE 4
Nonstrategy Medication Frequencies at 24 Months

| | No. (%) of Patients | | |
|---|---|---|---|
| | Calcium | Non-Calcium | |
| | Antagonist | Antagonist | |
| | Strategy | Strategy | |
| Drug Use | (n = 6793) | (n = 6822) | P Value* |
| Antihypertensive drug | | | |
| Any nonstudy | 2944 (43.3) | 2929 (42.9) | .64 |
| ACE inhibitor | 1300 (19.1) | 1310 (19.2) | .93 |
| Centrally acting | 132 (1.9) | 137 (2.0) | .79 |
| Calcium antagonist | 1133 (16.7) | 479 (7.0) | <.001 |
| Diuretic | 1314 (19.3) | 1439 (21.1) | .01 |
| §-Blocker/other vasodilator | 365 (5.4) | 365 (5.4) | .96 |
| †-Blocker | 373 (5.5) | 967 (14.2) | <.001 |
| Other class | 616 (9.1) | 626 (9.2) | .83 |
| Other drug | | | |
| Aspirin or other | 3938 (58.0) | 3905 (57.2) | .39 |
| antiplatelet agent | | | |
| Other NSAIDs | 1205 (17.7) | 1211 (17.8) | .99 |
| Antidiabetic medication† | 1574 (23.2) | 1682 (24.7) | .04 |
| Any lipid-lowering agent | 2836 (41.7) | 2822 (41.4) | .66 |
| Nitrates | 2024 (29.8) | 2116 (31.0) | .13 |
| Potassium supplement | 469 (6.9) | 507 (7.4) | .24 |
| Hormone replacement‡ | 601 (17.1) | 599 (16.9) | .81 |

Abbreviations:
ACE, angiotensin-converting enzyme;
NSAIDs, nonsteroidal anti-inflammatory drugs.
*Results from χ² test to compare strategies.
†Insulin and/or oral hypoglycemics.
‡Data for women only (n = 3520 for calcium antagonist strategy and n = 3554 for non-calcium antagonist strategy).

Blood Pressure and Heart Rate

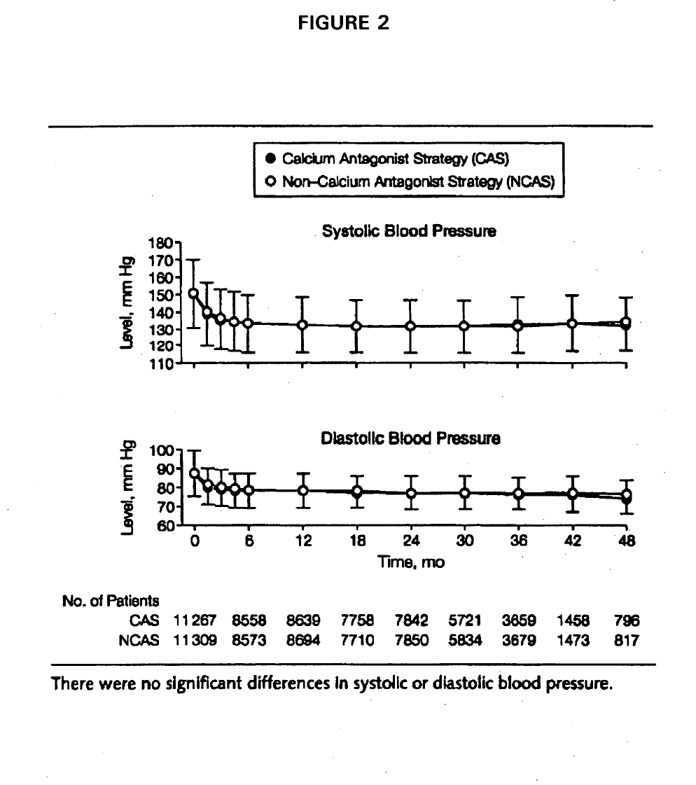

FIG. 2 presents SBP and DBP data by treatment strategy over 48 months (error bars in the upward direction represent 1 SD for CAS and bars in the downward direction represent 1 SD for NCAS). Mean (SD) SBP reduction at 24 months was 18.7 (22.2) mm Hg in the CAS group compared with 19.0 (22.6) mm Hg in the NCAS group (P=0.41). The mean (SD) DBP reduction at 24 months was 10.0 (12.4) mm Hg in the CAS group compared with 10.2 (12.4) mm Hg in the in the NCAS group (P=0.26). A reduction of 90% of the maximum achieved in SBP and 100% in DBP occurred in the first 6 months of treatment; the reductions were maintained throughout the trial. Mean resting heart rate at 24 months was significantly lower (P<0.001) in NCAS patients (69.2/mm) compared with CAS patients (72.8/min).

Outcomes

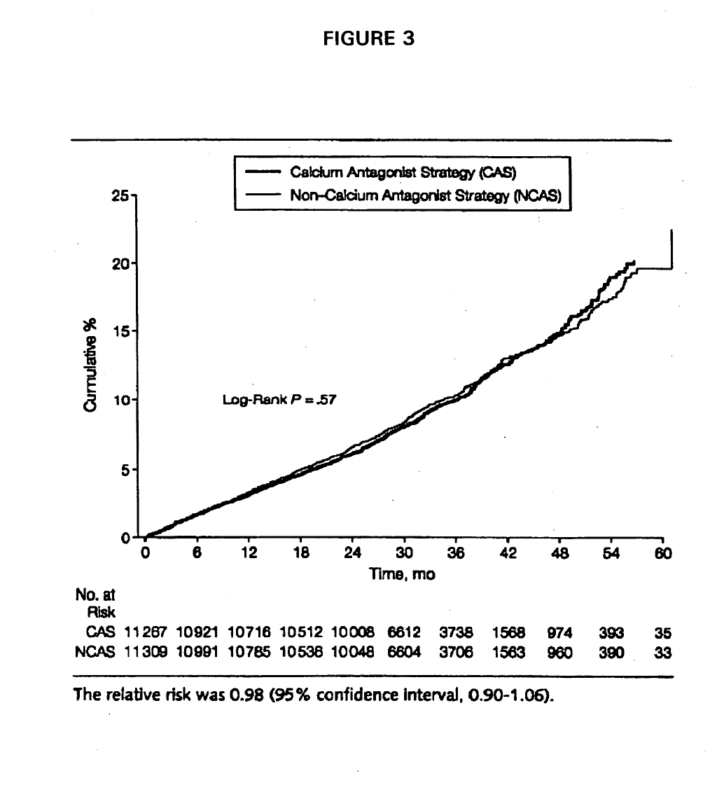

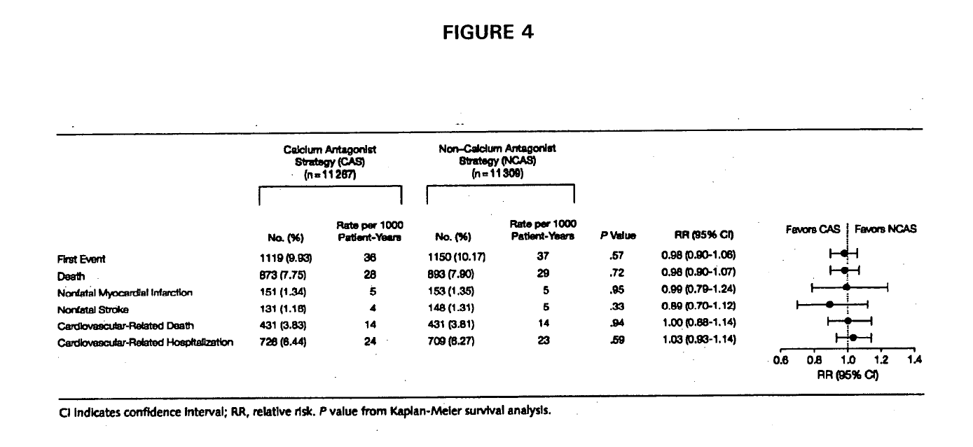

FIG. 3 presents the primary outcome and FIG. 4 presents the patients with events comprising the primary and other outcomes. A total of 2456 events were reported (CAS, 1214; NCAS, 1242) and the events committee confirmed 2380 of the events (96.9%; CAS, 1171; NCAS, 1209). Sites reported that 2333 patients (CAS, 1153; NCAS, 1180) experienced an event in the primary outcome cluster during follow-up and the events committee confirmed that an event had occurred in 2269 of those patients (97.3%; CAS, 1119; NCAS, 1150). Death (all-cause) occurred in 1766 patients (CAS, 873; NCAS, 893); 304 were non-fatal MIs (CAS, 151.; NCAS, 153); and 279 were nonfatal strokes (CAS, 131; NCAS, 148). Of the 1766 confirmed deaths, 862 were classified as definitely or presumed cardiovascular (CAS, 431; NCAS, 431); 701 were non-cardiovascular (CAS, 350; NCAS, 351); and 203 could not be classified (CAS, 92; NCAS, 111). Of the 1563 classifiable deaths, 862. (55.2%) were cardiovascular. The analyses reported herein were performed only on events confirmed by the events committee, but analyses of site-investigator reported events yielded similar results (data not shown).

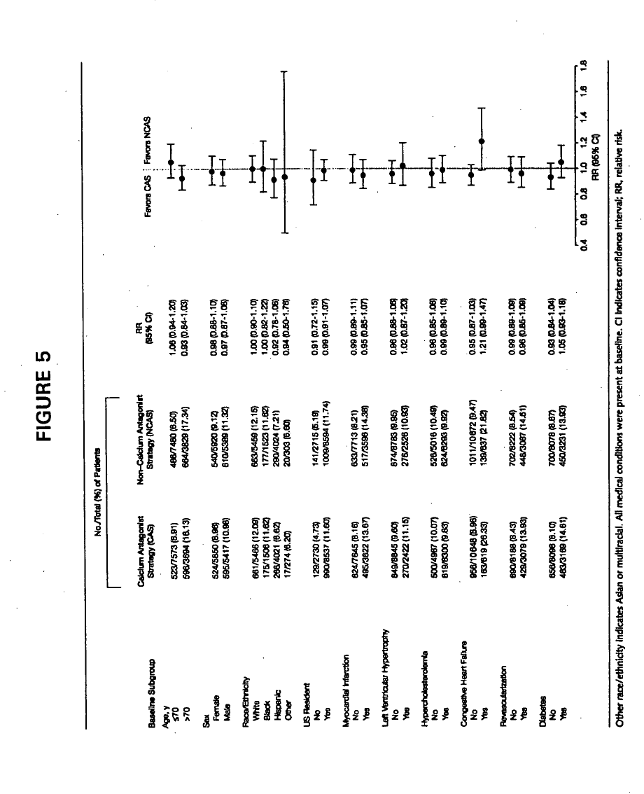

Kaplan-Meier analysis (unadjusted) of time to first primary outcome event demonstrated no difference comparing the CAS group with the NCAS group for a primary outcome (FIG. 3; RR, 0.98 [95% CI, 0.90-1.06]). A sensitivity analysis in which the 568 patients who were lost to follow-up or withdrew were all presumed to have died produced an RR of 1.00 (95% CI, 0.94-1.08). When adjusted for the pre-specified covariates of age, race, sex, previous MI, and previous heart failure, the CAS and NCAS groups were 0.98; 95% CI, 0.91-1.07; P=0.69). Other outcomes were also similar in frequency between strategies (FIG. 4). Time to death (all cause) did not differ between treatment groups (P=0.72), nor did time to nonfatal MI (P=95), or time to nonfatal stroke (P=0.33). Time to the most serious event also did riot differ between treatment groups (P=0.58). Fatal and nonfatal MI occurred in 452 CAS patients (4.01%) and 441 NCAS patients (3.90%) (RR, 1.03; 95% CI, 0.90-1.17). Fatal and nonfatal stroke occurred in 176 CAS patients (1.56%) and 201 NCAS patients (1.78%) (RR, 0.88; 95% CI, 0.72-1.07). Subgroup analyses by baseline characteristics showed consistency for the primary outcome in both high- and low-risk subgroups (FIG. 5). Of particular note were the similar event rates for each strategy among patients with prior MI as well as those with prior coronary revascularization. The exception was patients with prior heart failure, for which those assigned to the NCAS strategy appeared to have fewer events (P=0.03 for interaction). Also important was the marked difference in the event rate of 14.3% (913/6400) for those with diabetes compared with 8.4% (1356/16176) for those without diabetes.

The effect of the treatment strategies using an overall SBP control goal of less than 140 mm Hg anti DBP control goal of less than 90 mm Hg was similar. A total of 5625 patients (71.7%) in the CAS group and 5553 (70.7%) in the NCAS group achieved overall blood pressure control at 24 months (P=0.18). Based on JNC VI blood pressure goals, SBP control was achieved by 65.0% of CAS patients (n=5093) compared with 64.0% of NCAS patients (n=5025) (P=0.23); DBP control was achieved by 88.5% of CAS patients (n=6937) compared with 88.1% of NCAS patients (n=6914) (P=0.46).

At baseline, angina was reported in 66.2% of CAS patients (n=7463) compared with 67.0% of NCAS patients (n=7582). At 24 months, these percentages decreased to 27.3% in the CAS group (n=2055) and 28.3% in the NCAS group (n=2136) (P=0.18). Angina and unstable angina were infrequently reported as adverse experiences and rates were similar in both groups (TABLE 5). At baseline (based on the previous 4 weeks), there was a mean (SD) of 1.5 (2.33) angina episodes/wk in the CAS group and 1.5(2.43) in the NCAS group. At 24 months, angina episodes decreased in both groups, but the mean (SD) frequency was lower in the CAS group (0.77 [1.31] episodes/wk) compared with the NCAS group (0.88 [1.62] episodes/wk) (P=0.02). Revascularization was required in only 2% of patients in each group (TABLE 5). Nitrate use was the same in each strategy (TABLE 4).

TABLE 5
Adverse Experiences*

| | No. (%) of Patients | | |
|---|---|---|---|
| | Calcium | Non-Calcium | |
| | Antagonist | Antagonist | |
| | Strategy | Strategy | |
| Adverse Experience | (n = 11 267) | (n = 11 309) | P Value^{†} |
| Angina | 261 (2.32) | 228 (2.02) | .13 |
| CABG/PCI | 280 (2.49) | 275 (2.43) | .80 |
| Cancer | 192 (1.70) | 186 (1.64) | .73 |
| Constipation | 195 (1.73) | 15 (0.13) | <.001 |
| Cough | 201 (1.78) | 152 (1.34) | .01 |
| Dizziness | 154 (1.37) | 151 (1.34) | .84 |
| Dyspnea | 82 (0.73) | 114 (1.01) | .03 |
| Heart failure (class I-IV) | 189 (1.68) | 173 (1.53) | .38 |
| Lightheadedness | 48 (0.43) | 70 (0.62) | .05 |
| Symptomatic bradycardia | 74 (0.66) | 143 (1.26) | <.001 |
| Unstable angina | 131 (1.16) | 122 (1.08) | .55 |
| Wheezing | 17 (0.15) | 44 (0.39) | <.001 |
| other^{‡} | 1158 (10.28) | 1180 (10.43) | .70 |

Abbreviations:
CABG, coronary artery bypass graft;
PCI, percutaneous coronary intervention.
*Alzheimer disease, gastrointestinal tract bleeding, Parkinson disease, atrioventricular block, gout, headache, hyperkalemia, hypokalemia, abnormal liver enzymes, peripheral edema, peripheral vascular disease, renal failure, and transient ischemic attack were observed in 1% or fewer of each treatment group; differences between groups were nonsignificant (P > .05).
^{†}Results from χ² test for categorical measures.
^{‡}Adverse experiences that were not frequently reported and were not significantly different between strategies).

Analysis of the development of diabetes revealed significant differences between the treatment strategies. Of the 8098 CAS patients without diabetes at entry, 569 (7.03%) were diagnosed as having diabetes during follow-up. Of the 8078 NCAS patients without diabetes at entry, 665 (8.23%) were diagnosed as having diabetes during follow-up (RR, 0.85; 95% CI, 0.77-0.95). Patients in the CAS group were also less likely to die or develop diabetes compared with patients in the NCAS group (1050 [12.97%] vs 1177 [14.57%]; RR, 0.89; 95% CI, 0.82-0.96) and less likely to have an event in the primary outcome cluster or develop diabetes (1185 [14.63%] vs 1313 [16.25%]; RR, 0.90; 95% CI, 0.84-0.97). To explore possible explanations for reduced risk of diabetes, we conducted preliminary analyses adjusting for the 5 pre-specified baseline covariates (age, race, sex, prior MI, and prior heart failure) and included factors for average daily dose of add-on medication (trandolapril and/or hydrochlorothiazide). In these analyses, trandolapril appeared to confer a protective effect in the CAS group. Compared with those in the NCAS group not taking either trandolapril or hydrochlorothiazide, those in the CAS group not taking trandolapril had a HR of developing diabetes of 0.95 (95% CI, 0.82-1.10). A 2-mg dose of trandolapril was associated with a HR of 0.86 (95% CI, 0.74-1.00) and a 4-mg dose was associated with a HR of 0.77 (95% CI, 0.62-0.96). In the NCAS group, a 2-mg dose of trandolapril was associated with a HR of 0.99 (95% CI, 0.90-1.08) and a 4-mg dose was associated with a HR of 0.98 (95% CI, 0.82-1.18). On the other hand, hydrochlorothiazide appeared to confer a non-statistically significant increased risk of diabetes. Compared with those in the NCAS group not taking either trandolapril or hydrochlorothiazide, the addition of 12.5 mg of hydrochlorothiazide was associated with a HR of 1.17 (95% CI, 1.09-1.25) and 25 mg of hydrochlorothiazide was associated with a HR of 1.36 (95% CI, 1.18-1.57). Those in the CAS group not taking hydrochlorothiazide had a HR of 0.95 (95% CI, 0.82-1.10); the addition of 12.5 mg of hydrochlorothiazide was associated with a HR of 1.11 (95% CI, 0.95-1.29) and 25 mg of hydrochlorothiazide was associated with a HR of 1.28 (95% CI, 1.05-1.57).

Adverse Experiences

Both drug combinations were generally well tolerated in each treatment group. Cancer was reported in 192 patients (1.70%) in the CAS group compared with 186 patients (1.64%) in the NCAS group (P=0.73). Alzheimer disease, gastrointestinal tract bleeding, and Parkinson disease were reported in 1% or less of patients in each group and incidence did not differ between groups. Patients in the CAS group reported constipation and cough snore frequently than patients in the NCAS group, while NCAS patients had more dyspnea, lightheadedness, symptomatic bradycardia, and wheezing (TABLE 5).

Subanalysis

Subanalysis Design: INVEST afforded an opportunity to analyze unique and specific combinations of antihypertensive drugs due to the data accumulated for prescription date, drug, regimen, and dose. One subanalysis was performed to evaluate the incremental benefit of BP reduction by the addition of HCTZ to various dose combinations of verapamil SR and trandolapril in patients randomized to the calcium antagonist strategy. The dose combinations analyzed included HCTZ added to the following:
Group 1—Verapamil SR 180 mg/trandolapril 2 mg
Group 2—Verapamil SR 240 mg/trandolapril 2 mg
Group 3—Verapamil SR 180 mg/trandolapril 4 mg

Patients who received the verapamil SR/trandolapril combination as their first randomized drug prescription in INVEST, and who received no other drug prescription or gap in medication until their first HCTZ prescription added to the initial verapamil SR/trandolapril combination dose, were selected for this subgroup analysis. Because the dosing schedule in the study protocol recommended starting with verapamil SR as monotherapy, and investigators were free to vary doses as well as drugs prescribed during the trial, only a small proportion of patients satisfied these strict conditions. However, these sub-populations were selected as the cleanest available subgroup to attempt to estimate additional blood pressure reduction observed when HCTZ was added. Summary statistics for change from baseline for SBP and DBP to the last observation at each dose level for both the verapamil SR/trandolapril dual combination and the verapamil SR/trandolapril/HCTZ triple combination were analyzed. In INVEST, patients could have been treated with doses of HCTZ in the range of 12.5-100 mg. In this subanalysis the incremental effect of HCTZ on BP reduction was evaluated for any dose of HCTZ.

Baseline Characteristics: The baseline characteristics of the three verapamil SR/trandolapril/HCTZ groups are shown in TABLE 6. The groups were generally comparable with respect to gender and age. Group 1 had the highest baseline BP and the largest proportion of Blacks. Group 2 had the largest proportion of Hispanics. Group 3 had the highest weight and body mass index (BMI) and the largest proportion of Caucasians.

TABLE 6
Subanalysis Baseline Characteristics

| | Verapamil SR/Trandolapril Group (Dose) | | |
|---|---|---|---|
| | Group 1 | Group 2 | Group 3 |
| Parameter | (180/2 mg) | (240/2 mg) | 240/4 mg |
| N | 94 | 21 | 22 |
| Age, mean (years) | 64.5 | 62.1 | 64.1 |
| Female (%) | 55.3 | 52.4 | 45.5 |
| Weight, mean (kg) | 74.7 | 85.2 | 92.7 |
| BMI, mean (kg/m²) | 28.2 | 31.4 | 31.8 |
| Race (%) | | | |
| Caucasian | 45.7 | 42.9 | 86.4 |
| Black | 19.1 | 4.8 | 9.1 |
| Hispanic | 35.1 | 52.4 | 4.5 |
| Systolic BP, mean (mm Hg) | 164.4 | 160.3 | 160.7 |
| Diastolic BP, mean (mm Hg) | 96.2 | 90.0 | 84.6 |

Subanalysis Results: The results of incremental BP reduction with the addition of HCTZ to Groups 1, 2, and 3 are shown in TABLE 7. BP reductions after patients were prescribed the initial verapamil SR/trandolapril combinations were variable due to differences in the quantity and type of baseline antihypertensive medication. Because baseline antihypertensive medications were discontinued at randomization, their impact on the evaluation of HCTZ additivity was negligible. For each verapamil SR/trandolapril combination group, clinically significant BP reduction was observed with the addition of HCTZ. Decreases of mean systolic BP were 7.1, 6.5, and 3.2 mm Hg for Groups 1, 2, and 3, respectively, after the addition of HCTZ. Decreases of mean diastolic BP were 1.1, 4.2 and 2.3 mm Hg for Groups 1, 2, and 3, respectively, with the addition of HCTZ. Thus, the addition of HCTZ to various dose combinations of verapamil SR/trandolapril conferred further BP reduction in hypertensive patients.

TABLE 7
Changes in Systolic and Diastolic BP

| | | | | Verapamil SR |
|---|---|---|---|---|
| | | | Verapamil SR | Trandolapril |
| | | | Trandolapril | HCTZ |
| | BP Parameter | Baseline | (Dual Therapy) | (Triple Therapy) |
| Group 1 | Mean (SD) last | 164.4/96.2 | 149.3/85.9 | 142.2/84.8 |
| | observation (mm Hg) | (20.6/13.7) | (18.3/12.6) | (15.0/10.3) |
| | Mean change (SD) from | N/A | −15.0/−10.3 | −22.2/−11.4 |
| | baseline (mm Hg) | | (22.7/12.7) | (20.9/13.1) |
| | Mean change (SD) from | N/A | N/A | −7.1/−1.1 |
| | dual therapy (mm Hg) | | | (19.6/12.3) |
| Group 2 | Mean (SD) last | 160.3/90.0 | 153.6/88.4 | 147.1/84.3 |
| | observation (mm Hg) | (24.3/15.3) | (19.7/10.1) | (13.3/10.5) |
| | Mean change (SD) from | N/A | −6.7/−1.5 | −13.2/−5.7 |
| | baseline (mm Hg) | | (22.8/10.9) | (23.5/14.4) |
| | Mean change (SD) from | N/A | N/A | −6.5/−4.2 |
| | dual therapy (mm Hg) | | | (17.7/8.3) |
| Group 3 | Mean (SD) last | 160.7/84.6 | 149.9/81.7 | 146.7/79.3 |
| | observation (mm Hg) | (17.1/11.4) | (15.0/10.5) | (13.8/10.3) |
| | Mean (SD) change from | N/A | −10.8/−2.9 | −13.9/−5.3 |
| | baseline (mm Hg) | | (16.1/9.6) | (19.8/8.6) |
| | Mean change (SD) from | N/a | N/A | −3.2/−2.3 |
| | dual therapy (mm Hg) | | | (16.1/8.0) |

Group 1 - (Verapamil SR 180 mg/Trandolapril 2 mg) N = 94
Group 2 - (Verapamil SR 240 mg/Trandolapril 2 mg) N = 21
Group 3 - (Verapamil SR 240 mg/Trandolapril 4 mg) N = 22

Subanalysis Conclusion: The results of this subanalysis demonstrate that HCTZ confers clinically significant, incremental BP reduction in hypertensive patients treated with a combination of verapamil SR and trandolapril. Additional BP reduction was observed with HCTZ for each of the three verapamil SR/trandolapril combinations evaluated: 180/2 mg. 240/2 mg, and 240/4 mg. (TABLE 7, “Mean change (SD) from dual therapy (mm Hg)). Because many hypertensive patients require more than two agents to achieve BP goals, the verapamil SR/trandolapril/HCTZ triple combination could be a reasonable treatment option for moderate to severe hypertension.

Further, the subanalysis indicates that triple combination therapy with a 2 mg dose of trandolapril (Groups 1 and 2) decreases blood pressure with similar efficacy as triple combination therapy with a 4 mg dose of trandolapril (Group 3). Interestingly, the mean change from dual therapy was greater for Groups 1 and 2 (2 mg trandolapril) than it was for Group 3 (4 mg trandolapril). (See mean change from dual therapy for Groups 1, 2, and 3 in Table 7.) These results demonstrate that it is possible to achieve essentially equivalent reductions in blood pressure with half the dose of trandolapril in triple combination therapies, thereby avoiding any needless side-effects that may be observed with higher doses.

Conclusion

One objective of the INVEST study was to test the hypothesis that treatment of hypertensive CAD patients with either a verapamil SR-based strategy (CAS group) or a β-blocker-based strategy (atenolol; NCAS group) would result in equivalent clinical outcomes. The findings of the study demonstrated that these treatment strategies were equivalent in the prevention of the outcome of all-cause mortality, nonfatal MI, or nonfatal stroke. Furthermore, similar results were observed comparing the treatment strategies for all-cause mortality, cardiovascular death, cardiovascular hospitalization, and blood pressure control. Significant differences were observed between strategies that favored the verapamil SR plus trandolapril strategy (CAS group) for lower angina frequency and new diagnoses of diabetes. There was a significant interaction between treatment group and prior heart failure, suggesting that those randomized to the atenolol plus hydrochlorothiazide strategy (NCAS group) had better outcomes than those randomized to the verapamil SR plus trandolapril strategy (CAS group). Both strategies were well tolerated.

INVEST is the first, to our knowledge, large randomized, prospective trial to focus on CAD patients with hypertension and to follow JNC VI guidelines, which recommend use of an ACE inhibitor for special populations and lower blood pressure goals than other guidelines. It is important to note that this was not simply a comparison of verapamil SR with atenolol because it was anticipated that few patients would be treated with only those drugs. At study end, most were taking the combination of verapamil SR plus trandolapril (CAS group) or atenolol plus hydrochlorothiazide (NCAS group). Also, the study population included a high percentage of elderly, female, nonwhite, and diabetic patients. Thus, the results reported herein should be clinically applicable.

Although other trials have investigated use of calcium antagonists in hypertensive patients, the frequency of CAD in these trials was too low to reach any relevant conclusions. For example, the Nordic Diltiazem (NORDIL) study demonstrated equivalence between diltiazem and diuretics and/or β-blockers for cardiovascular morbidity and mortality and showed a reduction in incidence of fatal and nonfatal stroke in the diltiazem group, but only a small proportion of those patients (4.5%; n=496) had coronary heart disease. Results from several hypertension trials, including LIFE and ALLHAT, have been confounded by differences in achieved blood pressure level, which influences outcomes. In our study, the reductions and achieved levels for SBP and DBP were similar its both treatment groups. Most INVEST patients achieved JNC VI goals for blood pressure control. These findings in patients with CAD extend those from LIFE and ALLHAT, demonstrating that even lower blood pressure targets are achievable with more aggressive management. However, ALLHAT neither tested a β-blocker arm nor used an angiotensin II active agent for organ protection for patients with diabetes, renal impairment, or heart failure. Thus, INVEST results complement ALLHAT by including a β-blocker-based strategy plus organ protection in an elderly population with CAD. The INVEST data also confirm and extend the suggestions of others that monotherapy is not necessarily sufficient for optimal treatment of hypertension.

Overall, adverse experiences reported were minimal and similar in frequency between treatment strategies. Previous articles have suggested that some calcium antagonists (principally short-acting dihydropyridines) may be associated with an increased risk of cancer, gastrointestinal tract bleeding, and all-cause mortality. Results of ALLHAT, STOP-2, and INVEST have not confirmed these suggestions. The difference in crossover rates may reflect the consequences of adverse experiences (dyspnea, lightheadedness, symptomatic bradycardia, and wheezing) associated with the combination of atenolol plus hydrochlorothiazide (NCAS group) compared with adverse experiences (constipation and cough) associated with the combination of verapamil SR plus trandolapril (CAS group). The possibility that the higher crossover rate in the atenolol based strategy is related to previous intolerance or physician bias against β-blockers cannot be excluded, particularly because patients recently taking β-blockers were excluded from the trial. Another possibility is that the differing drug components of CAS (verapamil SR plus trandolapril) or NCAS (atenolol plus hydrochlorothiazide) could have conferred advantages in addition to blood pressure control. The combination of verapamil SR plus trandolapril could result in fewer metabolic complications, as was observed with reduction of new diagnoses of diabetes. The NCAS might have been expected to have advantages in patients with a prior MI and prior coronary revascularization; however, the results observed were similar with both strategies. Our outcome data for patients with prior heart failure, on the other hand, concur with recent trials documenting benefits of β-blockers when added to diuretics and ACE inhibitors, although not all patients in those trials had hypertension. In light of the results reported herein, management of hypertension must focus on the risk profile of the patient and overall treatment regimen rather than a single drug.

The study used blood pressure goals in accordance with JNC VI; however, JNC VII and epidemiological data indicate that CHD risk increases with SBP levels higher than 115 mm Hg, so it could be argued that even lower blood pressure targets may be reasonable. More than half the patients required 3 or more antihypertensive drugs to achieve blood pressure control. Better blood pressure control might have been possible if a fourth drug was included in each of the specified treatment strategies. The large sample size resulted in a statistically significant difference in angina frequency comparing CAS with NCAS, but this difference may not be clinically significant. The decline in angina prevalence and frequency from entry (only 2% underwent revascularization) is clinically important. This, at least in part, is likely due to the decline in both SBP and heart rate. Lastly, although the new diabetes analysis was not planned before the trial started, this outcome was added early in the recruitment phase. The findings suggest potential clinical implications that require confirmation. Other analyses of INVEST baseline data indicate that Hispanic ethnicity, heart failure, US residency, hypercholesterolemia, left ventricular hypertrophy, stroke and transient ischemic attack, prior coronary revascularization, and body mass index are linked to risk of developing diabetes. In the preliminary analyses herein, administration of trandolapril appeared to confer some protection, as suggested in previous studies of ACE inhibitors. Hydrochlorothiazide was associated with a non-significantly increased risk of developing diabetes, which is also consistent with previous studies (usually a thiazide diuretic with a β-blocker). Further analyses are required to better understand the complex interactions among drug, dose, and demographic factors. Patients' potassium levels were not collected in this study, so the role that hypokalemia may have played in precipitating hyperglycemia cannot be determined.

In conclusion, our results indicate that lower targets for blood pressure control can be achieved in most hypertensive patients with CAD using a multidrug strategy that includes administration of ACE inhibitors to patients with heart failure, diabetes, or renal impairment. The clinical equivalence of the CAS and NCAS groups in prevention of death, MI, or stroke supports the use of either strategy in clinically stable patients with GAD who require blood pressure control. The decision regarding which drug classes to use in specific CAD patients should be based on additional factors including adverse experiences, history of heart failure, diabetes risk, and the physician's best judgment. The possibility of delaying the emergence of a diabetes diagnosis with a CAS compared with an NCAS requires further investigation.

All references, patents, and/or applications cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compounds/compositions described herein as presently representative of preferred embodiments are exemplary and are not intended as limitations on the scope of the invention. Changes therein and other uses will occur to those skilled in the art, which are encompassed within the invention.

It will be readily apparent to one skilled in the art that varying substitutions and modifications may be made to the invention disclosed herein without departing from the scope and spirit of the invention. For example, a variety of different binding pairs can be utilized, as well as a variety of different therapeutic and diagnostic agents. Thus, such additional embodiments are within the scope of the present invention.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

### References 1. Punzi et al., The Treatment of Severe Hypertension with Trandolapril, Verapamil, and Hydrochlorothiazide, Journal of Human Hypertension (1997) 11, 477-481.

2. Skoularigis et al., Once-Daily Fixed Dose Combinations of Verapamil and Trandolapril in Black Patients with Mild to Moderate Hypertension: a New Choice for First Line Treatment, International Journal of Clinical Pharmacology and Therapeutics, Vol. 35, No. 2 (1997), 51-55.

3. Murray et al., Global Mortality, Disability, and the Contribution of Risk Factors: Global Burden of Disease Study, Lancet 1997; 349: 1436-42.

4. EUROASPIRE II Study Group. Lifestyle and Risk Factor Management and Use of Drug Therapies in Coronary Patients From 15 Countries; Principal Results From EUROASPIRE II Euro Heart Survey Programme. Eur Heart J 2001; 22: 554-72.

5. Murray et al., Effectiveness and Costs of Interventions to Lower Systolic Blood Pressure and Cholesterol: a Global and Regional Analysis on Reduction of Cardiovascular-Disease Risk, Lancet 2003; 361: 717-25.

6. Dahlöf et al., Morbidity and Mortality in the Swedish Trial in Old Patients with Hypertension (STOP-Hypertension), Lancet 1991; 338: 1281-5.

7. MRC Working Party. Medical Research Council Trial of Treatment of Hypertension in Older Adults: Principal Results, Br Med J 1992; 304: 405-12.

8. Collins et al., Blood Pressure, Antihypertensive Drug Treatment and the Risks of Stroke and of Coronary Heart Disease, Br Med Bull 1994; 50: 272-98.

9. Psaty et al., Health Outcomes Associated with Antihypertensive Therapies Used as First-Line Agents, JAMA 1997; 277:739-45.

10. Staessen et al., for the Systolic Hypertension in Europe (Syst-Eur) Trial Investigators. Randomised Double-Blind Comparison of Placebo and Active Treatment for Older Patients with Isolated Systolic Hypertension, Lancet 1997; 350: 757-64.

11. Pitt, et al., Effect of Amlodipine on the Progression of Atherosclerosis and the Occurrence of Clinical Events, Circulation 2000; 102: 1503-10

12. HOPE (Heart Outcomes Prevention Evaluation) Study Investigators. Effects on an Angiotensin-Converting-Enzyme Inhibitor, Ramipril, on Cardiovascular Events in High-Risk Patients, N Engl J Med 2000; 342: 145-53.

13. UK Prospective Diabetes Study Group, Tight Blood Pressure Control and Risk of Macrovascular and Microvascular Complications in Type 2 Diabetes: UKPDS 38, BMJ 1998; 317: 703-20.

14. UK Prospective Diabetes Study Group, Efficacy of Atenolol and Captopril in Reducing Risk of Macrovascular and Microvascular Complications in Type 2 Diabetes: UKPDS 39, BMJ 1998; 317: 713-20

15. Zanchetti et al., The Verapamil in Hypertension and Atherosclerosis Study (VHAS): Results of Long-Term Randomised Treatment with Either Verapamil or Chlorthalidone on Intima-Media Thickness, J Hyperten 1998; 16: 1667-76.

16. Arauz-Pacheco, et al., The Treatment of Hypertension in Adult Patients with Diabetes, Diabetes Care 2002; 25: 134-47.

17. Smith, Jr. et al., AHA/ACC Guidelines for Preventing Heart Attack and Death in Patients with Atherosclerotic Cardiovascular Disease: 2001 update, Circulation 2001; 104: 1577-79.

18. Marks R, et al., Enhancing Clinical Trials on the Internet: Lessons form INVEST, Clin cardiol 2001; 24 (11 suppl): V17-23.

19. Cooper-DeHoff R et al., Electronic Prescribing via the Internet for a Coronary Artery Disease and Hypertension Megatrial, Clin Cardiol 2001; 24 (11suppl): V14-V6.

20. Burt V L et al., Prevalence of Hypertension in the US Adult Population: Results From the Third National Health and Nutrition Examination Survey, 1988-1991. Hypertension 1995; 25: 305-13.

21. Sever, P S et al., for the ASCOT Investigators, Rationale, Design, Methods and Baseline Demography of Participants of the Anglo-Scandinavian Cardiac Outcomes Trial, J Hypertens 2001; 19: 1139-47.

22. American Heart Association. Heart Disease and Stroke Statistics-2003 Update. Dallas, Tex.: American Heart Association; 2002.

23. Chalmers J, MacMahon S. Mancia G, et al. Guidelines for the management of hypertension. Clin Exp. Hypertens. 1999;21:1009-1060.

24. Hansson L, Zanchetti I, Carruthers S C, et al. Effects of intensive blood-pressure lowering and low-dose aspirin in patents with hypertension. Lancet. 1998;351:1755-1762.

25. Probstfield J L, for the SHEP Cooperatve Research Group. Prevention of stroke by antihypertensive drug treatment in older persons with isolated systolic hypertension. JAMA. 1991:265:3255-3264.

26. Hansson L, Lindholm L H, Ekbom T, et al. Randomised trial of old and new antihypertensive drugs in elderly patients. Lancet. 1999;354:1751-1756.

27. Brown M J, Palmer C R, Castaigne A, et al. Morbidity and mortality in patients randomised to double-blind treatment with a long-acting calcium-channel blocker or diuretic in the International Nifedipine GITS study. Lancet. 2000;356:366-372.

28. Chobanian A V, Bakris C L, Black H R, et al. The Seventh Report of the Joint National Committee on Prevention, Detection, Evaluation, and Treatment of High Blood Pressure. JAMA. 2003;289:2560-2572.

29. Hansson L, Lindholm L H, Niskanen L, et al. Effect of angiotensin-converting-enzyme inhibition compared with conventional therapy on cardiovascular morbidity and mortality in hypertension. Lancet 1999;353:611-616.

30. The Sixth Report of the Joint National Committee on Prevention, Detection, Evaluation, and Treatment of High Blood Pressure. Arch Intern Med. 1997;157:2413-2446 [Erratum, Arch. Intern. Med. 1998;158:573].

31. Yusuf S, Sleight P, Pogue J, et al. Effects of an angiotensin-converting enzyme inhibitor, ramipril, on cardiovascular events in high-risk patients. N Engl J. Med. 2000:342:145-153.

32. Hennekens C H, Albert C M, Godfried S L, Gaziano J M, Buring J E. Adjunctive drug therapy for acute myocardial infarction—evidence from clinical trials. N Eng J. Med. 1996:335:1660-1667.

33. The Fifth Report of the Joint National Committee on Detection, Evaluation, and Treatment of High Blood Pressure (JNC V). Arch Intern Med. 1993;153:154-183.

34. Messerli F H, Grossman E, Goldbourt U. Are β-bloceers efficacious as first-line therapy for hypertension in the elderly? JAMA. 1998:279:1903-1907.

35. Dahlöf B, Devereux R B, Kjeldsen S E, et al. Cardiovascular morbidity and mortality in the Losartan Intervention For Endpoint Reduction in Hypertension study (LIFE): a randomised trial against atenolol. Lancet. 2002:359:995-1003.

36. Neal B, MacMahon S, Chapman N, et al. Effects of ACE inhibitors, calcium antagonists, and other blood-pressure-lowering drugs. Lancet. 2000:356:1955-1964.

37. ALLHAT Officers and Coordinators for the ALLHAT Collaborative Research Group. Major outcomes in high-risk hypertensive patients randomized to angiotensin-converting enzyme inhibitor or calcium channel blocker vs diuretic. JAMA. 2002:288:2981-2997.

38. Furberg C D, Psaty B M, MeyeriV. Nifedipine. Circulation. 1995:92:1326-1331.

39. Epstein M. The calcium antagonist controversy. Am J. Cardiol. 1997;79(suppl. 10A), 9-19.

40. Pepine C J. The role of calcium antagonists in ischaemic heart disease. Eur. Heart J. 1995;16(suppl H):19-24.

41. Pepine C J, Faich G, Makuch R. Verapamil use in patients with cardiovascular disease: an overview of randomized trials. Clin. Cardiol. 1998:21:633-641.

42. Hansson L, HednerT, Lund-Johansen P, et al. Randomised trial of effects of calcium antagonists compared with diuretics and β-blockers on cardiovascular morbidity and mortality in hypertension: the Nordic Diltiazem (NORDIL) study. Lancet. 2000;356:359-365.

43. Ruilope L M, de la Sierra A, Moreno E, et al. Prospective comparison of therapeutical attitudes in hypertensive type 2 diabetic patients uncontrolled on monotherapy. a randomized trial: the EDICTA study. J. Hypertens. 1999:17:1917-1923.

44. Karlberg B E, Andrup M, Oden A, for the Swedish TARKA trialists. Efficacy and safety of a new long-acting drug combination, trandolapril/verapamil as compared to monotherapy in primary hypertension. Blood Press. 2000:9:140-145.

45. PROCOPA Study Group. Dissociation between blood pressure reduction and fall in proteinuria in primary renal disease. J Hypertens. 2002;20:729-737.

46. Holzgreve H, Nakov R, Beck K, Janka H U. Anti-hypertensive therapy with verapamil SR plus trandolapril versus atenolol plus chlorthalidone on glycemic control. Am J. Hypertens. 2003:16:381-386.

47. Wing L M H, Reid C M, Ryan P, et al. A comparison of outcomes with angiotensin-converting enzyme inhibitors and diuretics for hypertension in the elderly. N Engl. J. Med. 2003:348:583-592.

48. Parving H H, Lehnert H, Bröchner-Mortensen J, et al. The effect of irbesartan on the development of diabetic nephropathy in patients with type 2 diabetes. N Engl J Med. 2001;345:870-878.

49. Brenner B M, Cooper M E, De Zeeuw D, et al. Effects of losartan on renal and cardiovascular outcomes in patients with type 2 diabetes and nephropathy. N Engl. J. Med. 2001:345:861-869.

50. Estacio R O, Jeffers B W, Hiatt W R, Biggerstaff S L, Gifford N, Schrier R W. The effect of nisoldipine as compared with enalapril on cardiovascular outcomes in patients with non-insulin-dependent diabetes and hypertension. N Engl. J. Med. 1998:338:645-652.

51. Wright T T Jr. Bakris C, Greene T, et al. Effect of blood pressure lowering and antihypertensive drug class on progression of hypertensive Kidney disease: results from the AASK trial. JAMA. 2002:19:2421-2431.

52. Pepine C J, Handberg-Thurmond E, Marks R G, et al. Rationale and design of the International Verapamil SR/Trandolapril Study (INVEST). J Am Coll Cardiol. 1998;32:1228-1237.

53. Hansson L, Hedner T, Dahlöf B. Prospective Randomized Open Blinded End-point (PROBE) study. Blood Press. 1992:1:113-119.

54. INVEST information pack. Available at: http://www.medicine.ufl.edu/cardio/research/invest_info/invest_definitions.shtml. Accessed Oct. 4, 2003.

55. Alderman M, Arakawa K, Beilin L, et al. Effects of calcium antagonists on the risks of coronary heart disease, cancer and bleeding. J. Hypertens. 1997:15:105-115.

56. Bakris G L. Williams M, Dworkin L, et al. Preserving renal functions in adults with hypertension and diabetes. Am J. Kidney Dis. 2000:36:646-661.

57. Psaty B M, Heckbert S R, Koepsell T O, et al. The risk of myocardial infarction associated with antihypertensive drug therapies. JAMA. 1995:274:620-625.

58. Pahor M, Psaty B M, Alderman M H, et al. Health outcomes associated with calcium antagonists compared with other first-line antihypertensive therapies. Lancet. 2000:356:1949-1954.

59. MERIT-HF Study Group. Effect of metoprolol CR/XL in chronic heart failure. Lancet. 1999:353:2001-2007.

60. Dargie H J, Lechat P. The Cardiac Insufficiency Bisoprolol Study II (CIBIS-II: a randomised trial. Lancet. 1999:353:9-13.

61. Packer M, Coats A J S, Fowler M B, et al. Effect of carvedilol on survival in severe chronic heart failure. N Engl J. Med. 2001:344:1651-1658.

62. Lewington S, Clarke R, QlzilbasH N, Peto R, Collins R. Age-specific relevance of usual blood pressure to vascular mortality. Lancet. 2002;360:1903-1913.

63. Cooper-DeHoff R, Bakris C, Cohen J, et al. Reduced risk of new onset diabetes in hypertensive coronary artery disease patients treated with a calcium antagonist vs a beta blocker regimen: the International Verapamal/Trandolapril Stxdy. Circulation. 2003:108:IV-750.

64. Wright J, Davis B, Cutler J, et al. Long-term cardiovascular consequences of diuretics vs calcium channel blockers vs angiotensin-converting enzyme inhibitors. JAMA. 2003:289:2067-2069.

65. Bengtsson C, Blohme C, Lapidus L, Lissner L, Lundgren H. Diabetes incidence in users and non-users of antihypertensive drugs in relation to serum insulin, glucose tolerance and degree of adiposity. J. Intern. Med. 1992:231:583-588.

66. Bakris G L, Weir M R, DeQuattro V, McMahon F G. Effects of an ACE inhibitor/calcium antagonist combination on proteinuria in diabetic nephropathy. Kidney Int 1998; 54: 1283-9.

67. Black H R, Elliott W J, Neaton J D, et al. Baseline characteristics and early blood pressure control in the CONVINCE trial. Hypertension 2001; 37: 12-8.

1. A pharmaceutical composition comprising:
between about 0.5 mg and about 2 mg trandolapril or a therapeutic derivative thereof;
verapamil or a therapeutic derivative thereof;
hydrochlorothiazide or a therapeutic derivative thereof; and
one or more pharmaceutically suitable carriers or excipients.

2. The composition of claim 1, wherein the composition comprises about 2 mg trandolapril or a therapeutic derivative thereof.

3. The composition of claim 1, wherein the composition comprises about 1 mg trandolapril or a therapeutic derivative thereof.

4. The composition of claim 1, wherein the composition comprises between about 40 mg and about 360 mg verapamil or a therapeutic derivative thereof.

5. The composition of claim 4, wherein the composition comprises between about 180 mg and about 240 mg verapamil or a therapeutic derivative thereof.

6. The composition of claim 5, wherein the composition comprises about 240 mg verapamil or a therapeutic derivative thereof.

7. The composition of claim 5, wherein the composition comprises about 180 mg verapamil or a therapeutic derivative thereof.

8. The composition of claim 1, wherein the composition comprises between about 6.25 mg and about 100 mg hydrochlorothiazide or a therapeutic derivative thereof.

9. The composition of claim 8, wherein the composition comprises between about 6.25 mg and about 25 mg hydrochlorothiazide or a therapeutic derivative thereof.

10. The composition of claim 1, wherein the composition comprises:
about 0.5, 1, or 2 mg trandolapril or a therapeutic derivative thereof;
about 40, 80, 100, 120, 180, 200, 220, 240, 300, or 360 mg verapamil or a therapeutic derivative thereof; and
about 6.25, 12.5, 25, 50, 75, or 100 mg hydrochlorothiazide or a therapeutic derivative thereof.

11. The composition of claim 10, wherein the composition comprises about 2 mg trandolapril or a therapeutic derivative thereof.

12. The composition of claim 10, wherein the composition comprises about 1 mg trandolapril or a therapeutic derivative thereof.

13. The composition of claim 10, wherein the composition comprises about 240 mg verapamil or a therapeutic derivative thereof.

14. The composition of claim 10, wherein the composition comprises about 180 mg verapamil or a therapeutic derivative thereof.

15. The composition of claim 10, wherein the composition comprises about 6.25, 12.5, or 25 mg hydrochlorothiazide or a therapeutic derivative thereof.

16. The composition of claim 10, wherein the composition comprises:
about 2 mg trandolapril or a therapeutic derivative thereof; and
about 240 mg verapamil or a therapeutic derivative thereof.

17. The composition of claim 16, wherein the composition comprises about 6.25, 12.5, or 25 mg hydrochlorothiazide or a therapeutic derivative thereof.

18. The composition of claim 10, wherein the composition comprises:
about 2 mg trandolapril or a therapeutic derivative thereof; and
about 180 mg verapamil or a therapeutic derivative thereof.

19. The composition of claim 18, wherein the composition comprises about 6.25, 12.5, or 25 mg hydrochlorothiazide or a therapeutic derivative thereof.

20. The composition of claim 1, wherein verapamil, trandolapril, and hydrochlorothiazide or their therapeutic derivatives are present as active ingredients for treating hypertension.

21. The composition of claim 20, further comprising a matrix formulated for controlled release of one or more of the active ingredients after the composition is administered to a patient.

22. The composition of claim 21, wherein one or more of the active ingredients provide effective blood pressure control for about 24-30 hours when administered to a patient.

23. The composition of claim 22, wherein the composition releases effective amounts of one or more of the active ingredients for about 24-30 hours to achieve a systolic blood pressure of no more than about 150-140 mm Hg and a diastolic blood pressure of no more than about 90 mm Hg in the patient.

24. The composition of claim 22, wherein the composition releases effective amounts of one or more of the active ingredients to achieve a decrease in systolic blood pressure and diastolic blood pressure of at least about 10% in the patient relative to baseline measurements.

25. The composition of claim 21, wherein the composition provides effective blood pressure control for about 1 to 30 days when administered to a patient.

26. The composition of claim 25, wherein the composition provides effective blood pressure control for about 7 days when administered to a patient.

27. The composition of claim 1, wherein the composition is formulated for oral, topical, transdermal, subcutaneous, parenteral, or pulmonary administration.

28. The composition of claim 27, wherein the composition is formulated for oral, transdermal, or parenteral administration.

29. The composition of claim 1, wherein the composition is formulated as one or more tablets, capsules, granules, powders, liquids, suspensions, or emulsions.

30. A pharmaceutical composition for treating hypertension, comprising one or more pharmaceutically suitable carriers or excipients and active ingredients including:
trandolapril or a therapeutic derivative thereof, present in an amount between about 0.5 mg and about 2 mg;
verapamil or a therapeutic derivative thereof; and
hydrochlorothiazide or a therapeutic derivative thereof;
wherein one or more of the active ingredients are present in an amount to achieve effective blood pressure control in a patient to which the composition is administered.

31. The composition of claim 30, wherein the composition comprises about 2 mg trandolapril or a therapeutic derivative thereof.

32. The composition of claim 30, wherein the composition comprises about 1 mg trandolapril or a therapeutic derivative thereof.

33. The composition of claim 30, wherein verapamil or a therapeutic derivative thereof is present in an amount between about 40 mg and about 360 mg.

34. The composition of claim 33, wherein verapamil or a therapeutic derivative thereof is present in an amount between about 180 mg and about 240 mg.

35. The composition of claim 34, wherein the composition comprises about 240 mg verapamil.

36. The composition of claim 35, wherein the composition comprises about 180 mg verapamil.

37. The composition of claim 30, wherein one or more of the active ingredients are present in an amount to achieve a systolic blood pressure of no more than about 150-140 mm Hg and a diastolic blood pressure of no more than about 90 mm Hg in a patient to which the composition is administered.

38. The composition of claim 30, wherein one or more of the active ingredients are present in an amount to achieve a systolic blood pressure of no more than about 135 mm Hg and a diastolic blood pressure of no more than about 85 mm Hg in a patient to which the composition is administered.

39. The composition of claim 30, wherein one or more of the active ingredients are present in an amount to achieve a systolic blood pressure of no more than about 130 mm Hg and a diastolic blood pressure of no more than about 80 mm Hg in a patient to which the composition is administered.

40. The composition of claim 30, wherein one or more of the active ingredients are present in an amount to achieve a decrease in systolic blood pressure and diastolic blood pressure of at least about 10% relative to baseline measurements in a patient to which the composition is administered.

41. The composition of claim 30, wherein the patient has coronary artery disease.

42. The composition of claim 30, wherein the patient has renal disease.

43. The composition of claim 30, wherein the patient has diabetes or is at risk for acquiring diabetes.

44. The composition of claim 30, wherein the patient is about 60 years of age or older.

45. The composition of claim 44, wherein the patient is about 70 years of age or older.

46. A method for treating hypertension in a patient with coronary artery disease comprising administering a composition which includes one or more pharmaceutically suitable carriers or excipients and active ingredients including:
an angiotensin-converting-enzyme inhibitor;
a calcium channel blocker; and
a diuretic;
wherein one or more of the active ingredients are present in an effective amount to treat hypertension and the method achieves and maintains effective blood pressure control in the patient.

47. The method of claim 46, wherein the method achieves and maintains a systolic blood pressure of no more than about 150-140 mm Hg and a diastolic blood pressure of no more than about 90 mm Hg in the patient.

48. The method of claim 47, wherein the method achieves and maintains a systolic blood pressure of no more than about 135 mm Hg and a diastolic blood pressure of no more than about 85 mm Hg in the patient.

49. The method of claim 48, wherein the method achieves and maintains a systolic blood pressure of no more than about 130 mm Hg and a diastolic blood pressure of no more than about 80 mm Hg in the patient.

50. The method of claim 46, wherein the method achieves and maintains a decrease in systolic blood pressure and diastolic blood pressure of at least about 10% relative to baseline measurements in the patient.

51. The method of claim 46, wherein the angiotensin-converting-enzyme inhibitor is trandolapril or a therapeutic derivative thereof, the calcium channel blocker is verapamil or a therapeutic derivative thereof, and the diuretic is hydrochlorothiazide or a therapeutic derivative thereof.

52. The method of claim 51, wherein the composition comprises between about 0.5 and about 2 mg trandolapril.

53. The method of claim 52, wherein the composition comprises about 2 mg trandolapril.

54. The method of claim 52, wherein the composition comprises about 1 mg trandolapril.

55. The method of claim 52, wherein the composition comprises between about 40 mg and about 360 mg of verapamil; and between about 6.25 mg and about 100 mg of hydrochlorothiazide.

56. The method of claim 55, wherein the composition comprises about 240 mg of verapamil.

57. The method of claim 55, wherein the composition comprises about 180 mg of verapamil.

58. The method of claim 46, wherein the patient has diabetes.

59. The method of claim 46, wherein the patient has renal disease.

60. The method of claim 46, wherein the patient is about 60 years of age or older.

61. The method of claim 60, wherein the patient is about 70 years of age or older.

62. A method for decreasing mortality in a patient with coronary artery disease comprising administering a composition which comprises one or more pharmaceutically suitable carriers or excipients and active ingredients including:
an angiotensin-converting-enzyme inhibitor;
a calcium channel blocker; and
a diuretic;
wherein one or more of the active ingredients are present in an effective amount to treat hypertension and the method achieves and maintains effective blood pressure control in the patient.

63. A method for decreasing the likelihood of a heart attack and/or a myocardial infarction in a patient with coronary artery disease comprising administering a composition which comprises one or more pharmaceutically suitable carriers or excipients and active ingredients including:
an angiotensin-converting-enzyme inhibitor;
a calcium channel blocker; and
a diuretic;
wherein one or more of the active ingredients are present in an effective amount to treat hypertension and the method achieves and maintains effective blood pressure control in the patient.

64. A method of decreasing the likelihood of a stroke in a patient with coronary artery disease comprising administering a composition which comprises one or more pharmaceutically suitable carriers or excipients and active ingredients including:
an angiotensin-converting-enzyme inhibitor;
a calcium channel blocker; and
a diuretic;
wherein one or more of the active ingredients are present in an effective amount to treat hypertension and the method achieves and maintains effective blood pressure control in the patient.

65. A method of decreasing the likelihood of acquiring diabetes or delaying the onset of diabetes in a patient with coronary artery disease comprising administering a composition which comprises one or more pharmaceutically suitable carriers or excipients and active ingredients including:
an angiotensin-converting-enzyme inhibitor;
a calcium channel blocker; and
a diuretic;
wherein one or more of the active ingredients are present in an effective amount to treat hypertension and the method achieves and maintains effective blood pressure control in the patient.

66. A kit for treating hypertension in a patient with coronary artery disease comprising: (a) one or more dosage units, wherein a dosage unit comprises one or more pharmaceutically acceptable excipients and active ingredients including an angiotensin-converting-enzyme inhibitor, a calcium antagonist, and a diuretic; and one or more of the active ingredients are present in an effective amount to treat hypertension, and (b) instructions for using the kit to treat, prevent, and/or ameliorate hypertension by achieving and maintaining effective blood pressure control in the patient.

67. The kit of claim 66, wherein the kit is used to achieve and maintain a systolic blood pressure of no more than about 150-140 mm Hg and a diastolic blood pressure of no more than about 90 mm Hg in the patient.

68. The kit of claim 66, wherein the kit is used to achieve and maintain a decrease in systolic blood pressure and diastolic blood pressure of at least about 10% relative to baseline measurements in the patient.

69. The kit of claim 66, wherein the angiotensin-converting-enzyme inhibitor is trandolapril or a therapeutic derivative thereof; the calcium antagonist is verapamil or a therapeutic derivative thereof; and the diuretic is hydrochlorothiazide or a therapeutic derivative thereof.

70. The kit of claim 69, wherein the dosage unit comprises between about 0.5 mg and about 2 mg trandolapril.

71. The kit of claim 70, wherein the dosage unit comprises about 2 mg trandolapril.

72. The kit of claim 70, wherein the dosage unit comprises about 1 mg trandolapril.

73. The kit of claim 70, wherein the dosage unit comprises between about 40 mg and about 360 mg verapamil; and between about 6.25 mg and about 100 mg hydrochlorothiazide.

74. The kit of claim 73, wherein the dosage unit comprises about 240 mg verapamil.

75. The kit of claim 73, wherein the dosage unit comprises about 180 mg verapamil.

76. The kit of claim 66, wherein the kit comprises up to about 35 dosage units.

77. The kit of claim 66, further comprising instructions for using the kit to treat coronary artery disease, to decrease mortality, to decrease the likelihood of a heart attack and/or myocardial infarction, to decrease the likelihood of acquiring diabetes, and/or to decrease the likelihood of a stroke.

78. A kit for treating hypertension in a patient with coronary artery disease comprising:
(a) active ingredients formulated as:
(i) one or more dosage units comprising an angiotensin-converting-enzyme inhibitor and one or more pharmaceutically suitable carriers or excipients;
(ii) one or more dosage units comprising a calcium antagonist and one or more pharmaceutically suitable carriers or excipients; and
(iii) one or more dosage units comprising a diuretic and one or more pharmaceutically suitable carriers or excipients;
wherein one or more of the dosage units comprise the active ingredient in an effective amount to treat hypertension in the patient; and
(b) instructions for using the kit to treat, prevent, and/or ameliorate hypertension by achieving and maintaining effective blood pressure control in the patient.

79. The kit of claim 78, wherein the kit is used to achieve and maintain a systolic blood pressure of no more than about 150-140 mm Hg and a diastolic blood pressure of no more than about 90 mm Hg in the patient.

80. The kit of claim 78, wherein the kit is used to achieve and maintain a decrease in systolic blood pressure and diastolic blood pressure of at least about 10% relative to baseline measurements in the patient.

81. The kit of claim 78, wherein the angiotensin-converting-enzyme inhibitor is trandolapril or a therapeutic derivative thereof; the calcium antagonist is verapamil or a therapeutic derivative thereof; and the diuretic is hydrochlorothiazide or a therapeutic derivative thereof.

82. The kit of claim 81, wherein the trandolapril dosage unit comprises between about 0.5 mg and about 2 mg trandolapril.

83. The kit of claim 82, wherein the trandolapril dosage unit comprises about 2 mg trandolapril.

84. The kit of claim 83, wherein the trandolapril dosage unit comprises about 1 mg trandolapril.

85. The kit of claim 82, wherein the verapamil dosage unit comprises between about 40 mg and about 360 mg verapamil; and the hydrochlorothiazide dosage unit comprises between about 6.25 mg and about 100 mg hydrochlorothiazide.

86. The kit of claim 85, wherein the verapamil dosage unit comprises about 240 mg verapamil.

87. The kit of claim 85, wherein the verapamil dosage unit comprises about 180 mg verapamil.

88. The kit of claim 78, further comprising instructions for using the kit to treat coronary artery disease, to decrease mortality, to decrease the likelihood of a heart attack and/or myocardial infarction, to decrease the likelihood of acquiring diabetes, and/or to decrease the likelihood of a stroke.
